(19) United States
(12) Patent Application Publication
(10) Pub. No.: US 2009/0038623 A1
(43) Pub. Date: Feb. 12, 2009

(54) INDUCTIVE POWER TRANSFER SYSTEM FOR PALATAL IMPLANT

(75) Inventors: John Michael Farbarik, Castro Valley, CA (US); Anthony Nguyen, Milpitas, CA (US); Anant V. Hegde, Hayward, CA (US); Casidy Domingo, San Mateo, CA (US); Charisse Yung, Los Altos Hills, CA (US); Nikhil D. Bhat, Fremont, CA (US); George Yoseung Choi, Redwood City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pavad Medical, Inc., Fremont, CA (US)

(21) Appl. No.: 12/192,924

(22) Filed: Aug. 15, 2008

Related U.S. Application Data
(63) Continuation-in-part of application No. 11/613,027, filed on Dec. 19, 2006.
Application No. 11/613,027 is a continuation-in-part of application No. 11/355,927, filed on Feb. 15, 2006.
Application No. 11/355,927 is a continuation-in-part of application No. 11/233,493, filed on Sep. 21, 2005.
Application No. 11/233,493 is a continuation-in-part of application No. 10/946,435, filed on Sep. 21, 2004.

(51) Int. Cl. A61F 5/56; H02J 7/00

(52) U.S. Cl. 128/848; 320/108

(57) ABSTRACT

An inductive power transfer system associated with an airway implant device is disclosed. A non-implanted portion of the system comprises a mouthpiece or retainer. The mouthpiece includes a power transfer circuit and a power source. The power transfer circuit includes a receive circuit configured to receive a first inductive power transfer from a charging device and to deliver a charging current to the power source. The power transfer circuit also includes a transmit circuit coupled to the power source. The transmit circuit is configured to provide a second inductive power transfer from the mouthpiece to the airway implant. Some embodiments of the non-implanted portion include a charge controller and a lithium polymer battery. Some embodiments of the charging device include a microcontroller for controlling operation of the charging device based on a proximity of the mouthpiece.

### CROSS-REFERENCE

This application is a continuation in part of U.S. patent application Ser. No. 11/613,027 (atty. docket no. 026705-000312) filed Dec. 19, 2006 which is a continuation in part of U.S. patent application Ser. Nos. 10/946,435, filed Sep. 21, 2004, 11/233,493 filed Sep. 21, 2005, and 11/355,927 filed Feb. 15, 2006, all of which are incorporated herein by reference in their entirety.

### BACKGROUND OF THE INVENTION

Snoring is very common among mammals including humans. Snoring is a noise produced while breathing during sleep due to the vibration of the soft palate and uvula. Not all snoring is bad, except it bothers the bed partner or others near the person who is snoring. If the snoring gets worse over time and goes untreated, it could lead to apnea.

Those with apnea stop breathing in their sleep, often hundreds of times during the night. Usually apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed.

In a given night, the number of involuntary breathing pauses or “apneic events” may be as high as 20 to 60 or more per hour. These breathing pauses are almost always accompanied by snoring between apnea episodes. Sleep apnea can also be characterized by choking sensations.

Sleep apnea is diagnosed and treated by primary care physicians, pulmonologists, neurologists, or other physicians with specialty training in sleep disorders. Diagnosis of sleep apnea is not simple because there can be many different reasons for disturbed sleep.

The specific therapy for sleep apnea is tailored to the individual patient based on medical history, physical examination, and the results of polysomnography. Medications are generally not effective in the treatment of sleep apnea. Oxygen is sometimes used in patients with central apnea caused by heart failure. It is not used to treat obstructive sleep apnea.

Nasal continuous positive airway pressure (CPAP) is the most common treatment for sleep apnea. In this procedure, the patient wears a mask over the nose during sleep, and pressure from an air blower forces air through the nasal passages. The air pressure is adjusted so that it is just enough to prevent the throat from collapsing during sleep. The pressure is constant and continuous. Nasal CPAP prevents airway closure while in use, but apnea episodes return when CPAP is stopped or it is used improperly. Many variations of CPAP devices are available and all have the same side effects such as nasal irritation and drying, facial skin irritation, abdominal bloating, mask leaks, sore eyes, and headaches. Some versions of CPAP vary the pressure to coincide with the person's breathing pattern, and other CPAPs start with low pressure, slowly increasing it to allow the person to fall asleep before the full prescribed pressure is applied.

Dental appliances that reposition the lower jaw and the tongue have been helpful to some patients with mild to moderate sleep apnea or who snore but do not have apnea. A dentist or orthodontist is often the one to fit the patient with such a device.

Some patients with sleep apnea may need surgery. Although several surgical procedures are used to increase the size of the airway, none of them is completely successful or without risks. More than one procedure may need to be tried before the patient realizes any benefits. Some of the more common procedures include removal of adenoids and tonsils (especially in children), nasal polyps or other growths, or other tissue in the airway and correction of structural deformities. Younger patients seem to benefit from these surgical procedures more than older patients.

Uvulopalatopharyngoplasty (UPPP) is a procedure used to remove excess tissue at the back of the throat (tonsils, uvula, and part of the soft palate). The success of this technique may range from 30 to 60 percent. The long-term side effects and benefits are not known, and it is difficult to predict which patients will do well with this procedure.

Laser-assisted uvulopalatoplasty (LAUP) is done to eliminate snoring but has not been shown to be effective in treating sleep apnea. This procedure involves using a laser device to eliminate tissue in the back of the throat. Like UPPP, LAUP may decrease or eliminate snoring but not eliminate sleep apnea itself. Elimination of snoring, the primary symptom of sleep apnea, without influencing the condition may carry the risk of delaying the diagnosis and possible treatment of sleep apnea in patients who elect to have LAUP. To identify possible underlying sleep apnea, sleep studies are usually required before LAUP is performed.

Somnoplasty is a procedure that uses RF to reduce the size of some airway structures such as the uvula and the back of the tongue. This technique helps in reducing snoring and is being investigated as a treatment for apnea.

Tracheostomy is used in persons with severe, life-threatening sleep apnea. In this procedure, a small hole is made in the windpipe and a tube is inserted into the opening. This tube stays closed during waking hours and the person breathes and speaks normally. It is opened for sleep so that air flows directly into the lungs, bypassing any upper airway obstruction. Although this procedure is highly effective, it is an extreme measure that is rarely used.

Patients in whom sleep apnea is due to deformities of the lower jaw may benefit from surgical reconstruction. Surgical procedures to treat obesity are sometimes recommended for sleep apnea patients who are morbidly obese. Behavioral changes are an important part of the treatment program, and in mild cases behavioral therapy may be all that is needed. Overweight persons can benefit from losing weight. Even a 10 percent weight loss can reduce the number of apneic events for most patients. Individuals with apnea should avoid the use of alcohol and sleeping pills, which make the airway more likely to collapse during sleep and prolong the apneic periods. In some patients with mild sleep apnea, breathing pauses occur only when they sleep on their backs. In such cases, using pillows and other devices that help them sleep in a side position may be helpful.

Recently, Restore Medical, Inc., Saint Paul, Minn. has developed a new treatment for snoring and apnea, called the Pillar technique. Pillar System is a procedure where 2 or 3 small polyester rod devices are placed in the patient's soft palate. The Pillar System stiffens the palate, reduces vibration of the tissue, and prevents the possible airway collapse. Stiff implants in the soft palate, however, could hinder patient's normal functions like speech, ability to swallow, coughing and sneezing. Protrusion of the modified tissue into the airway is another long-term concern.

As the current treatments for snoring and/or apnea are not effective and have side-effects, there is a need for additional treatment options.

### BRIEF SUMMARY

An inductive power transfer system associated with an airway implant device is disclosed. A non-implanted portion of the system comprises a mouthpiece or retainer. The mouthpiece includes a power transfer circuit and a power source. The power transfer circuit includes a receive circuit configured to receive a first inductive power transfer from a charging device and to deliver a charging current to the power source. The power transfer circuit also includes a transmit circuit coupled to the power source. The transmit circuit is configured to provide a second inductive power transfer from the mouthpiece to the airway implant. Some embodiments of the non-implanted portion include a charge controller and a lithium polymer battery. Some embodiments of the charging device include a microcontroller for controlling the operation of the charging device based on a proximity of the mouthpiece.

In one embodiment, the receive circuit comprises a pickup coil and the transmit circuit comprises a power transmission coil. The non-implanted portion may also include a timer coupled to the receive circuit and the transmit circuit and configured to activate the transmit circuit a predetermined time after the first inductive transfer is complete. In some embodiments, the timer comprises a resistor and a capacitor and a voltage associated with the capacitor is used to define the predetermined interval.

In one embodiment, the non-implanted portion comprises a charge controller coupled to the receive circuit and the battery. The charge controller is configured to deliver a charging current to the battery. The charge controller optionally has a first operating mode in which the charging current is delivered to the battery at a substantially constant level, and a second operating mode in which the charging current is delivered to the battery so as to maintain the battery at a substantially constant voltage.

In one embodiment, a charging device is disclosed. The charging device has a housing to receive a mouthpiece. The charging device also includes a power transfer circuit configured to source an inductive power transfer when the mouthpiece is received at the housing. The power transfer circuit includes a power transmission coil. A proximity detection circuit is also included. The proximity detection circuit is configured to detect a proximity of the mouthpiece to the power transmission coil and to generate an output signal based on the proximity. The charging device initiates the inductive power transfer when the output signal exceeds a predetermined threshold.

In one embodiment, a device for powering an airway implant is disclosed. The device includes a non-implanted portion adapted and configured to be worn in a patient's mouth. A power transfer circuit is attached to the non-implanted portion. The power transfer circuit comprises a receive circuit configured to receive a first inductive power transfer from a charging device and to charge a battery with a current induced in the receive circuit by the charging device. The power transfer circuit also includes a transmit circuit coupled to the battery and configured to provide a second inductive power transfer from the non-implanted portion to the airway implant. A first microprocessor is coupled to the power transfer circuit and configured to control the operation of the transmit and receive circuits.

In one embodiment, a method for powering an airway implant device is disclosed. The airway implant device includes a non-implanted portion and an implant portion. The method includes receiving a first inductive power transfer at the non-implanted portion and charging a power source of the non-implanted portion with a current derived from the first inductive power transfer. The method also includes performing a second inductive transfer from the non-implanted portion to the implant portion. The power source supplies energy for the second inductive power transfer.

In one embodiment, an inductive power transfer system is disclosed. The system includes a prosthesis and a receiver implanted in a body cavity. The receiver is coupled to the prosthesis. The system also includes a wearable transmitter comprising a power source and a timer. The power source is configured to supply a first inductive power transfer to the receiver for operating the prosthesis. The timer is coupled to the power source and configured to define an interval during which the first inductive power transfer is disabled.

A method of treating a patient suffering from sleep disordered breathing is disclosed. The method includes placing an implant device in the patient's upper airway passage and inductively powering the implant device with an implanted receiver. A wearable transmitter supplies a first inductive power transfer to the implanted receiver and the implanted receiver supplies a second inductive power transfer to the implant device.

### BRIEF DESCRIPTION OF THE DRAWINGS

FIG. 1 illustrates one embodiment of the airway implant device.

FIG. 2 illustrates one embodiment of the airway implant device.

FIG. 3 illustrates one embodiment of the airway implant device.

FIG. 4 illustrates one embodiment of the airway implant device.

FIG. 5 illustrates a circuit diagram of an embodiment of the airway implant device.

FIG. 6 illustrates an embodiment of the airway implant device.

FIG. 7 illustrates a sectional view of an embodiment of the electroactive polymer element.

FIG. 8 illustrates a sectional view of an embodiment of the electroactive polymer element.

FIG. 9 illustrates an embodiment of the electroactive polymer element.

FIG. 10 illustrates an embodiment of the electroactive polymer element.

FIG. 11 illustrates an embodiment of the electroactive polymer element.

FIG. 12 illustrates an embodiment of the electroactive polymer element.

FIG. 13 illustrates an embodiment of the electroactive polymer element.

FIG. 14 illustrates an embodiment of the electroactive polymer element.

FIG. 15 illustrates an embodiment of the electroactive polymer element.

FIG. 16 illustrates an embodiment of the electroactive polymer element.

FIG. 17 illustrates an embodiment of the electroactive polymer element.

FIG. 18 illustrates an embodiment of the electroactive polymer element.

FIG. 19 illustrates an embodiment of the electroactive polymer element.

FIG. 20 illustrates an embodiment of the implanted portion of the airway implant device.

FIG. 21 illustrates an embodiment of the airway implant device.

FIG. 22 illustrates an embodiment of the non-implanted portion in the form of a mouth guard.

FIG. 23 illustrates an embodiment of the non-implanted portion in the form of a mouth guard.

FIG. 24 illustrates an embodiment of the non-implanted portion.

FIG. 25 shows a sagittal section through a head of a subject illustrating an embodiment of a method for using the airway implant device.

FIG. 26 illustrates an anterior view of the mouth with see-through mouth roofs to depict an embodiment of a method for using the airway implant device.

FIG. 27 illustrates an anterior view of the mouth with see-through mouth roofs to depict an embodiment of a method for using the airway implant device.

FIG. 28 illustrates an anterior view of the mouth with see-through mouth roofs to depict an embodiment of a method for using the airway implant device.

FIG. 29 illustrates an anterior view of the mouth with see-through mouth roofs to depict an embodiment of a method for using the airway implant device.

FIG. 30 illustrates an embodiment of an inductive coupling system associated with the airway implant device.

FIG. 31 illustrates an embodiment of the airway implant device.

FIG. 32 illustrates an embodiment of the airway implant device.

FIG. 33 illustrates an embodiment in which a patient wears the non-implanted portion of the device on the cheeks.

FIG. 34A-34B illustrates an embodiment of a method of the invention with the airway implant in the soft palate.

FIG. 35A-35B illustrates an embodiment of a method of the invention with the airway implants in the soft palate and lateral pharyngeal walls.

FIG. 36A-36B illustrates an embodiment of a method of the invention with the airway implants in the lateral pharyngeal walls.

FIG. 37 depicts the progression of an apneic event.

FIG. 38 depicts an embodiment of an airway implant device with sensors in the soft palate and laryngeal wall.

FIG. 39 depicts the functioning of an airway implant device with sensors in the soft palate and laryngeal wall.

FIG. 40 depicts an embodiment of an airway implant device with a sensor in the laryngeal wall.

FIG. 41 depicts an example of controller suitable for use with an airway implant device.

FIG. 42 depicts an embodiment of an airway implant device.

FIG. 43 depicts an embodiment of an airway implant device.

FIGS. 44A, 44B, and 44C illustrate terms used in describing the anatomy of a patient and orientation attributes of the invention.

FIG. 45A illustrates an embodiment of the airway implant device.

FIG. 45B illustrates the airway implant device of FIG. 45A, viewed from the anterior side of the implant, looking toward the posterior end, wherein the implant device is implanted in the palate.

FIG. 46A illustrates an embodiment of the airway implant device.

FIG. 46B illustrates the airway implant device of FIG. 46A, viewed from the anterior side of the implant, looking toward the posterior end, wherein the implant device is implanted in the palate.

FIG. 47A illustrates an embodiment of the airway implant device with a T-shaped attachment element.

FIG. 47B illustrates an embodiment of the airway implant device with a perforated attachment element.

FIG. 48 illustrates an embodiment of the airway implant device with saw-blade like directional attachment element.

FIG. 49 illustrates an embodiment of the airway implant device with power connecting element.

FIG. 50 illustrates an embodiment of the airway implant system with both an implantable device and a non-implantable wearable element.

FIG. 51A illustrates an isometric view of the wearable element.

FIG. 51B illustrates a bottom view of the wearable element.

FIG. 52 illustrates a cross-sectional view of the airway implant system in the patient soft palate.

FIG. 53 illustrates one embodiment of an inductive power transfer system associated with the airway implant device.

FIG. 54 illustrates an embodiment of a charging device.

FIG. 55 illustrates a mouthpiece holder such as can be used with the charging device of FIG. 54.

FIG. 56 illustrates an exemplary charging device.

FIG. 57 illustrates one embodiment of a microcontroller.

FIG. 58 shows exemplary processing states of a charging device.

FIG. 59 is a flowchart illustrating aspects of event log processing.

FIG. 60 illustrates an embodiment of the non-implanted portion.

FIG. 61 illustrates a further embodiment of the non-implanted portion.

FIG. 62 illustrates yet another embodiment of the non-implanted portion.

FIG. 63 shows aspects of an exemplary charging cycle.

### DETAILED DESCRIPTION

### Devices and Methods

A first aspect of the invention is a device for the treatment of disorders associated with improper airway patency, such as snoring or sleep apnea. The device comprises of an actuator element to adjust the opening of the airway. In a preferred embodiment, the actuator element comprises of an electroactive polymer (EAP) element. The electroactive polymer element in the device assists in maintaining appropriate airway opening to treat the disorders. Typically, the EAP element provides support for the walls of an airway, when the walls collapse, and thus, completely or partially opens the airway.

The device functions by maintaining energized and non-energized configurations of the EAP element. In preferred embodiments, during sleep, the EAP element is energized with electricity to change its shape and thus modify the opening of the airway. Typically, in the non-energized configuration the EAP element is soft and in the energized configuration is stiffer. The EAP element of the device can have a pre-set non-energized configuration wherein it is substantially similar to the geometry of the patient's airway where the device is implanted.

In some embodiments, the device, in addition to the EAP element, includes an implantable transducer in electrical communication with the EAP element. A conductive lead connects the EAP element and the implantable transducer to the each other. The device of the present invention typically includes a power source in electrical communication with the EAP element and/or the implantable transducer, such as a battery or a capacitor. The battery can be disposable or rechargeable.

Preferred embodiments of the invention include a non-implanted portion, such as a mouthpiece, to control the implanted EAP element. The mouthpiece is typically in conductive or inductive communication with an implantable transducer. In one embodiment, the mouthpiece is a dental retainer with an induction coil and a power source. The dental retainer can further comprise a pulse-width-modulation circuit. When a dental retainer is used it is preferably custom fit for the individual biological subject. If the implantable transducer is in inductive communication, it will typically include an inductive receiver, such as a coil. The implantable transducer can also include a conductive receiver, such as a dental filling, a dental implant, an implant in the oral cavity, an implant in the head or neck region. In one embodiment, the device includes a dermal patch with a coil, circuit and power source, in communication with the implantable transducer. The dermal patch can also include a pulse-width-modulation circuit.

Another aspect of the invention is a method to modulate air flow through airway passages. Such modulation is used in the treatment of diseases such as snoring and sleep apnea. One method of the invention is a method for modulating the airflow in airway passages by implanting in a patient a device comprising an actuator element and controlling the device by energizing the actuator element. The actuator element preferably comprises an electroactive polymer element. The actuator element can be controlled with a mouthpiece inserted into the mouth of the patient. The energizing is typically performed with the use of a power source in electrical communication, either inductive communication or conductive communication, with the actuator element. A transducer can be used to energize the actuator element by placing it in electrical communication with the power source. Depending on the condition being treated, the actuator element is placed in different locations such as soft palate, airway sidewall, uvula, pharynx wall, trachea wall, larynx wall, and/or nasal passage wall.

A preferred embodiment of the device of the present invention comprises an implantable actuator element; an implantable transducer; an implantable lead wire connecting the actuator element and the transducer; a removable transducer; and a removable power source; and wherein the actuator element comprises an electroactive polymer.

Electroactive polymer is a type of polymer that responds to electrical stimulation by physical deformation, change in tensile properties, and/or change in hardness. There are several types of electroactive polymers like dielectric electrostrictive polymer, ion exchange polymer and ion exchange polymer metal composite (IPMC). The particular type of EAP used in the making of the disclosed device can be any of the aforementioned electroactive polymers.

Suitable materials for the electroactive polymer element include, but are not limited to, an ion exchange polymer, an ion exchange polymer metal composite, an ionomer base material. In some embodiments, the electroactive polymer is perfluorinated polymer such as polytetrafluoroethylene, polyfluorosulfonic acid, perfluorosulfonate, and polyvinylidene fluoride. Other suitable polymers include polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate, polyvinyl pyrrolidone. Typically, the electroactive polymer element includes a biocompatible conductive material such as platinum, gold, silver, palladium, copper, and/or carbon.

Suitable shapes of the electroactive polymer element include three dimensional shape, substantially rectangular, substantially triangular, substantially round, substantially trapezoidal, a flat strip, a rod, a cylindrical tube, an arch with uniform thickness or varying thickness, a shape with slots that are perpendicular to the axis, slots that are parallel to the longitudinal axis, a coil, perforations, and/or slots.

IPMC is a polymer and metal composite that uses an ionomer as the base material. Ionomers are types of polymers that allow for ion movement through the membrane. There are several ionomers available in the market and some of the suited ionomers for this application are polyethylene, polystyrene, polytetrafluoroethylene, polyvinylidene fluoride, polyfluorosulfonic acid based membranes like NAFION® (from E. I. Du Pont de Nemours and Company, Wilmington, Del.), polyaniline, polyacrylonitrile, cellulose, cellulose acetates, regenerated cellulose, polysulfone, polyurethane, or combinations thereof. A conductive metal, for example gold, silver, platinum, palladium, copper, carbon, or combinations thereof, can be deposited on the ionomer to make the IPMC. The IPMC element can be formed into many shapes, for example, a strip, rod, cylindrical tube, rectangular piece, triangular piece, trapezoidal shape, arch shapes, coil shapes, or combinations thereof. The IPMC element can have perforations or slots cut in them to allow tissue in growth.

The electroactive polymer element has, in some embodiments, multiple layers of the electroactive polymer with or without an insulation layer separating the layers of the electroactive polymer. Suitable insulation layers include, but are not limited to, silicone, polyurethane, polyimide, nylon, polyester, polymethylmethacrylate, polyethylmethacrylate, neoprene, styrene butadiene styrene, or polyvinyl acetate.

In some embodiments, the actuator element, the entire device, or portions of the airway implant have a coating. The coating isolates the coated device from the body fluids and/or tissue either physically or electrically. The device can be coated to minimize tissue growth or promote tissue growth. Suitable coatings include poly-L-lysine, poly-D-lysine, polyethylene glycol, polypropylene, polyvinyl alcohol, polyvinylidene fluoride, polyvinyl acetate, hyaluronic acid, and/or methylmethacrylate.

### Embodiments of the Device

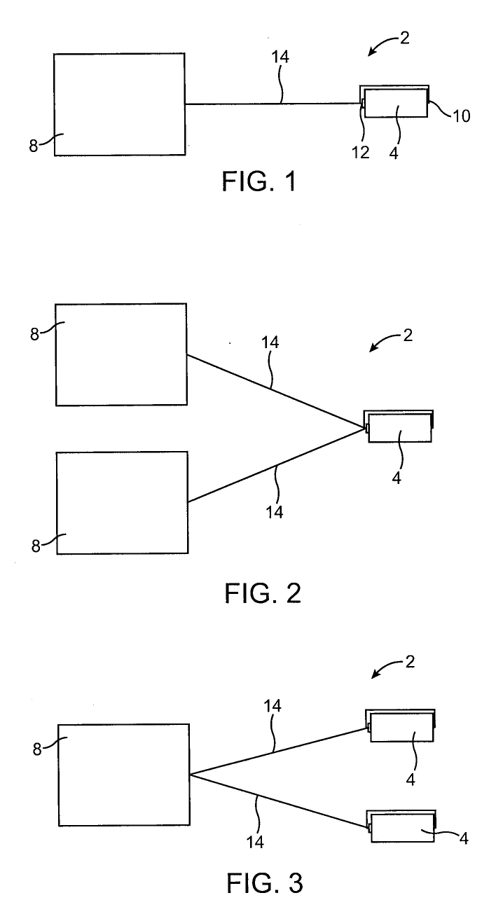

FIG. 1 illustrates an airway implant system 2 that has a power source 4, a connecting element, such as a wire lead 14, and an actuator element, such as an electroactive polymer element 8. Suitable power sources 4 are a power cell, a battery, a capacitor, a substantially infinite bus (e.g., a wall outlet leading to a power generator), a generator (e.g., a portable generator, a solar generator, an internal combustion generator), or combinations thereof. The power source 4 typically has a power output of from about 1 mA to about 5 A, for example about 500 mA.

Instead of or in addition to wire lead 14, the connecting element may be an inductive energy transfer system, a conductive energy transfer system, a chemical energy transfer system, an acoustic or otherwise vibratory energy transfer system, a nerve or nerve pathway, other biological tissue, or combinations thereof. The connecting element is made from one or more conductive materials, such as copper. The connecting element is completely or partially insulated and/or protected by an insulator, for example polytetrafluoroethylene (PTFE). The insulator can be biocompatible. The power source 4 is typically in electrical communication with the actuator element 8 through the connecting element. The connecting element is attached to an anode 10 and a cathode 12 on the power source 4. The connecting elements can be made from one or more sub-elements.

The actuator element 8 is preferably made from an electroactive polymer. Most preferably, the electroactive polymer is an ion exchange polymer metal composite (IPMC). The IPMC has a base polymer embedded, or otherwise appropriately mixed, with a metal. The IPMC base polymer is preferably perfluoronated polymer, polytetrafluoroethylene, polyfluorosulfonic acid, perfluorosulfonate, polyvinylidene fluoride, hydrophilic polyvinylidene fluoride, polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate and polyvinyl pyrrolidone, or combinations thereof. The IPMC metal can be platinum, gold, silver, palladium, copper, carbon, or combinations thereof.

FIG. 2 illustrates that the actuator element 8 can have multiple elements 8 and connecting elements 14 that all connect to a single power source 4.

FIG. 3 illustrates an airway implant system 2 with multiple power sources 4 and connecting elements 14 that all connect to a single actuator element 8. The airway implant system 2 can have any number and combination of actuator elements 8 connected to power sources 4.

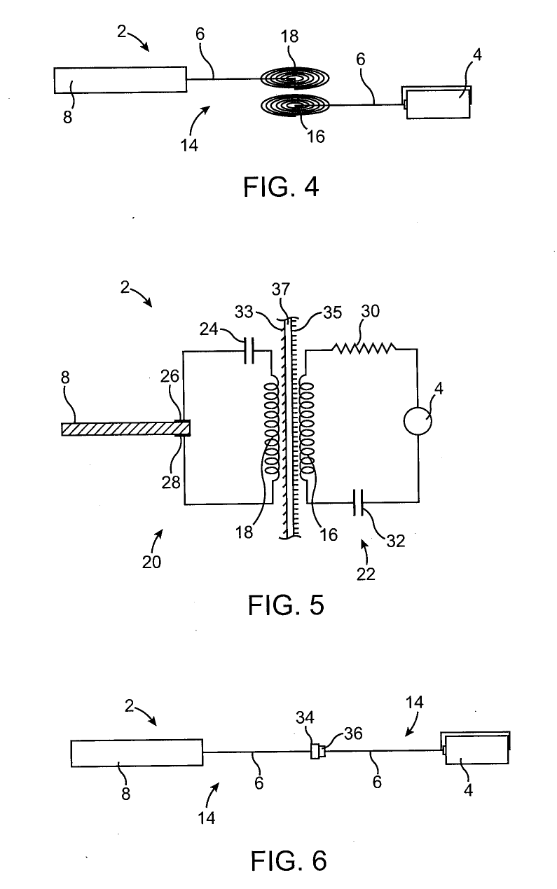

FIG. 4 illustrates an embodiment with the connecting element having a first energy transfer element, for example a first transducer such as a first receiver, and a second energy transfer element, for example a second transducer such as a second inductor 16. In this embodiment, the first receiver is a first inductor 18. The first inductor 18 is typically positioned close enough to the second inductor 16 to enable sufficient inductive electricity transfer between the second and first inductors 16 and 18 to energize the actuator element 8. The connecting element 14 has multiple connecting elements 6.

FIG. 5 illustrates that the airway implant device of the present invention can have an implanted portion 20 and a non-implanted portion 22. In this embodiment, the implanted portion 20 is a closed circuit with the first inductor 18 in series with a first capacitor 24 and the actuator element 8. The actuator element 8 is attached to the closed circuit of the implanted portion 20 by a first contact 26 and a second contact 28. In some embodiments, the implanted portion has a resistor (not shown). The non-implanted portion 22 is a closed circuit. The non-implanted portion 22 has a second inductor 16 that is in series with a resistor 30, the power source 4, and a second capacitor 32. The capacitors, resistors, and, in-part, the inductors are representative of the electrical characteristics of the wire of the circuit and not necessarily representative of specific elements. The implanted portion 20 is within tissue and has a tissue surface 33 nearby. The non-implanted portion is in insulation material 35. An air interface 37 is between the tissue surface 33 and the insulation material 35.

FIG. 6 illustrates an embodiment in which the first energy transfer element of the connecting element 14 is a first conductor 34. The second energy transfer element of the connecting element 14 is a second conductor 36. The first conductor 34 is configured to plug into, receive, or otherwise make secure electrical conductive contact with the second conductor 36. The first conductor 34 and/or second conductor 36 are plugs, sockets, conductive dental fillings, tooth caps, fake teeth, or any combination thereof.

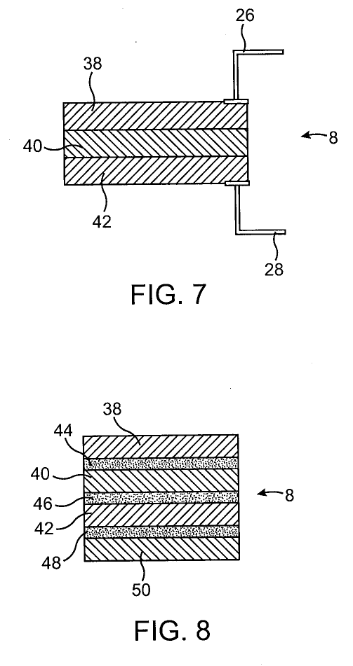

FIG. 7 illustrates an embodiment in which the actuator element 8 is a multi-layered device. The actuator element 8 has a first EAP layer 38, a second EAP layer 40, and a third EAP layer 42. The EAP layers 38, 40 and 42 are in contact with each other and not separated by an insulator.

FIG. 8 illustrates another embodiment in which the actuator element 8 has a first EAP layer 38 separated from a second EAP layer 40 by a first insulation layer 44. A second insulation layer 46 separates the second EAP layer from the third EAP layer 42. A third insulation layer 48 separates the third EAP layer from the fourth EAP layer 50. Insulation material is preferably a polymeric material that electrically isolates each layer. The insulation can be, for example, acrylic polymers, polyimide, polypropylene, polyethylene, silicones, nylons, polyesters, polyurethanes, or combinations thereof. Each EAP layer, 38, 40, 42 and 50 can be connected to a lead wire (not shown). All anodes and all cathodes are connected to the power source 4.

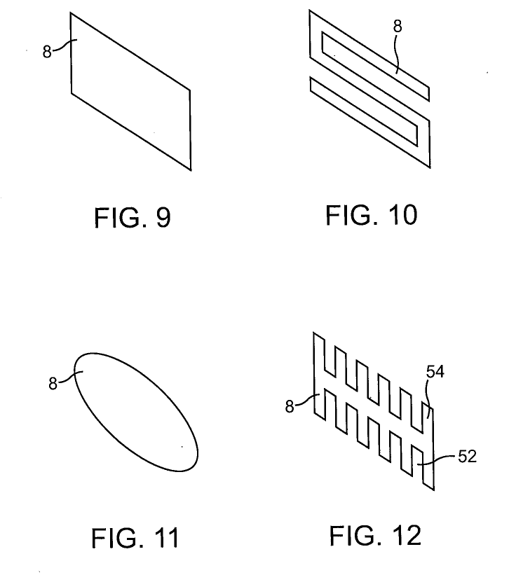

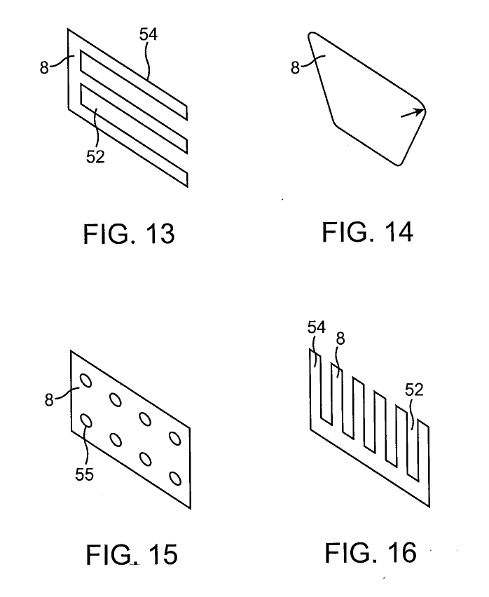

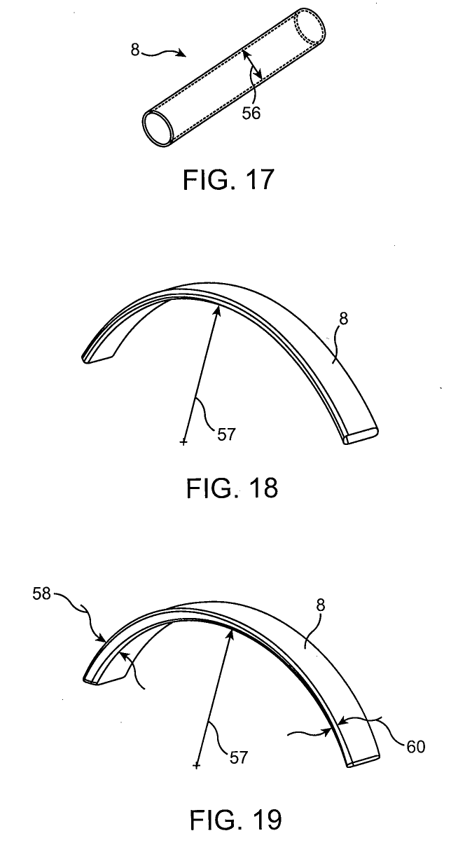

FIGS. 9-19 illustrate different suitable shapes for the actuator element 8. FIG. 9 illustrates a actuator element 8 with a substantially flat rectangular configuration. The actuator element 8 can have a width from about 2 mm to about 5 cm, for example about 1 cm. FIG. 10 illustrates an actuator element 8 with an “S” or zig-zag shape. FIG. 11 illustrates the actuator element 8 with an oval shape. FIG. 12 illustrates a actuator element 8 with a substantially flat rectangular shape with slots 52 cut perpendicular to the longitudinal axis of the actuator element 8. The slots 52 originate near the longitudinal axis of the actuator element 8. The actuator element 8 has legs 54 extending away from the longitudinal axis. FIG. 13 illustrates an actuator element 8 with slots 52 and legs 54 parallel with the longitudinal axis. FIG. 14 illustrates an actuator element be configured as a quadrilateral, such as a trapezoid. The actuator element 8 has chamfered corners, as shown by radius. FIG. 15 illustrates an actuator element 8 with apertures 55, holes, perforations, or combinations thereof. FIG. 16 illustrates an actuator element 8 with slots 52 and legs 54 extending from a side of the actuator element 8 parallel with the longitudinal axis. FIG. 17 illustrates an actuator element 8 with a hollow cylinder, tube, or rod. The actuator element has an inner diameter 56. FIG. 18 illustrates an arched actuator element 8. The arch has a radius of curvature 57 from about 1 cm to about 10 cm, for example about 4 cm. The actuator element 8 has a uniform thickness. FIG. 19 illustrates an arched actuator element 8. The actuator element 8 can have a varying thickness. A first thickness 58 is equal or greater than a second thickness 60.

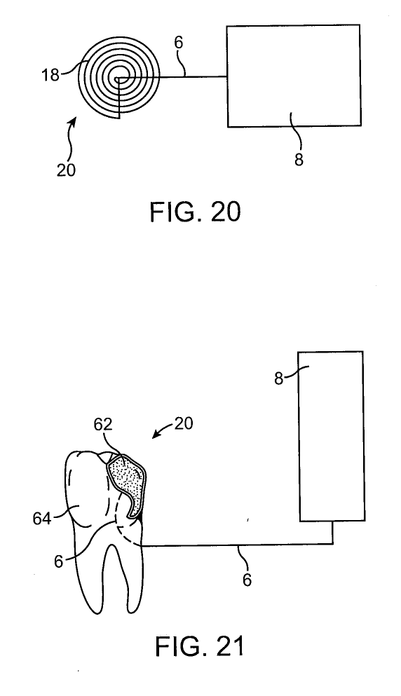

FIG. 20 illustrates an embodiment of the implanted portion of an airway implant with a coil-type inductor 18 connected by a wire lead 6 to the actuator element 8. In another embodiment, as illustrated in FIG. 21 the implanted portion has a conductive dental filling 62 in a tooth 64. The dental filling 62 is previously implanted for reasons related or unrelated to using of the airway implant system. The dental filling 62 is electrically connected to the wire lead 6. For example, a portion of the wire lead 6 is implanted in the tooth 64, as shown by phantom line. The wire lead 6 is connected to the actuator element 8.

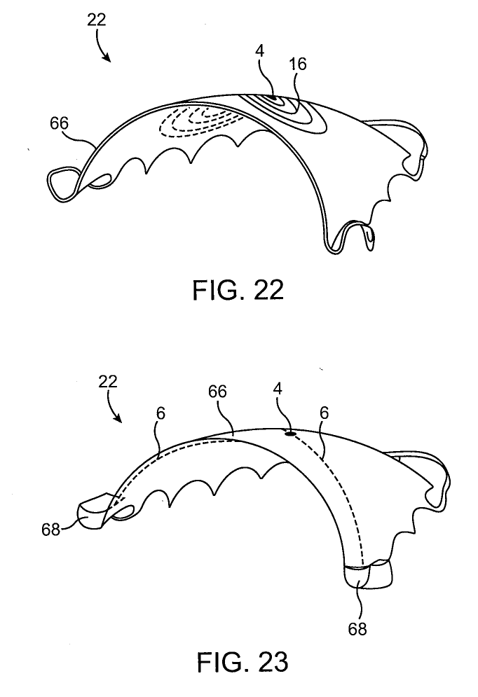

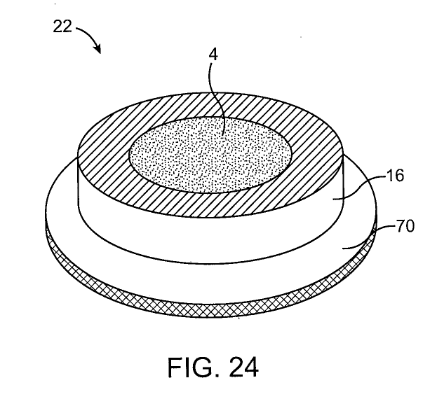

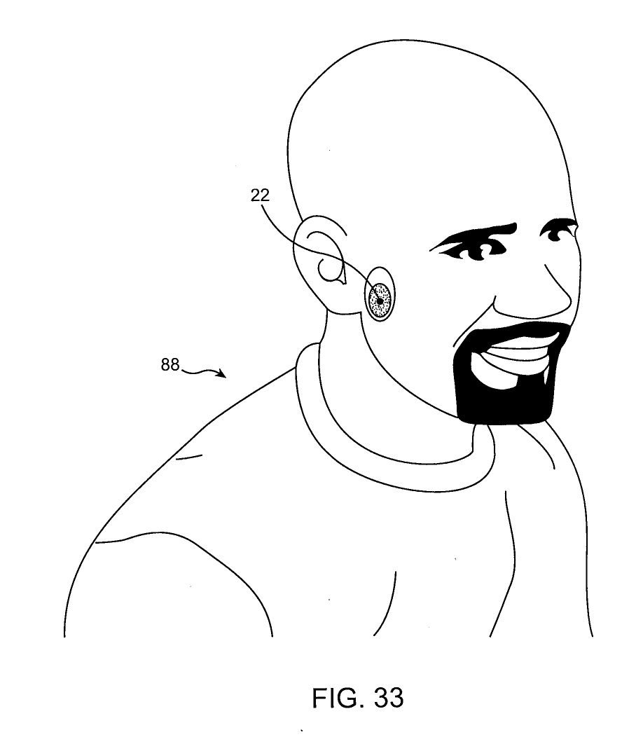

FIG. 22 illustrates an embodiment of the non-implanted portion 22 with a mouthpiece, such as a retainer 66. The retainer 66 is preferably custom configured to fit to the patient's mouth roof, or another part of the patient's mouth. The second transducer, such as second inductor 16, is integral with, or attached to, the retainer 66. The second inductor 16 is located in the retainer 66 so that during use the second inductor 16 is proximal with the first inductor 18. The power source 4, such as a cell, is integral with, or attached to, the retainer 66. The power source 4 is in electrical communication with the second inductor 16. In some embodiments, the retainer 66 has a pulse-width-modulation circuit. FIG. 23 illustrates that the retainer 66 has one or more tooth sockets 68. The tooth sockets 68 are preferably configured to receive teeth that have dental fillings. The tooth sockets 68 are electrically conductive in areas where they align with dental fillings when in use. The power source 4 is connected with the tooth sockets 68 via the wire leads 6. In the embodiment of FIG. 24, the non-implantable portion 22 has the second inductor 16 attached to a removably attachable patch 70. The patch 70 is attached to the power source 4. The power source 4 is in contact with the second inductor 16. This embodiment can be, for example, located on the cheeks as shown on FIG. 33 or any other suitable location.

Preferably, the airway implant device 2 discussed herein is used in combination with an inductive coupling system 900 such as depicted in FIG. 30. FIG. 30 depicts an inductive coupling system that is suitable for controlling the airway implant device 2 which includes a connecting element 906 (which connects the electrical contacts (not shown) to the rest of the electrical system), a connector 901, a energy source 322, a sensor 903, a timer 904, and a controller 905. The connector 901, energy source 322, sensor 903, a timer 904, and controller 905 are located in a housing disposed in a region outside or inside the body.

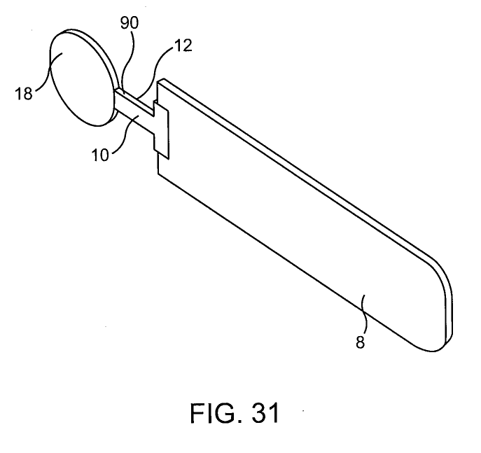

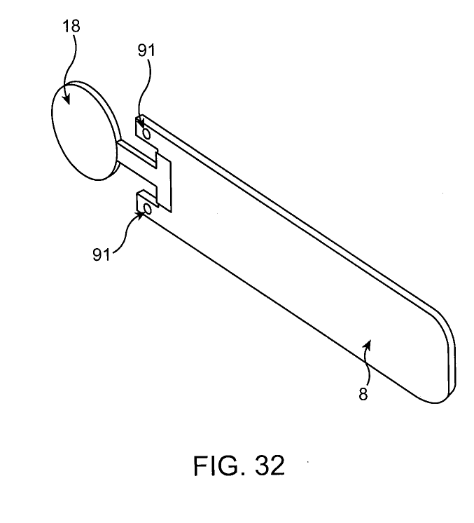

Two preferred embodiments of the airway implant device are shown in FIGS. 31 and 32. The device in FIG. 31 includes the actuator element 8 connected to an anode 10 and cathode 12 and to the induction coil 18. The device also includes a controller 90, such as a microprocessor. The circuitry within the controller is not shown. The controller 90 picks up AC signals from the induction coil 18 and converts it to DC current. The controller 90 can also include a time delay circuit and/or a sensor. The sensor could sense the collapsing and/or narrowing of the airways and cause the device to energize the actuator element 8 and thus completely or partially open up the airway in which the device is implanted. FIG. 32 shows an embodiment with anchors 91 located on the actuator element 8. The implant can be anchored in a suitable location with the use of these anchors and sutures and/or surgical glue.

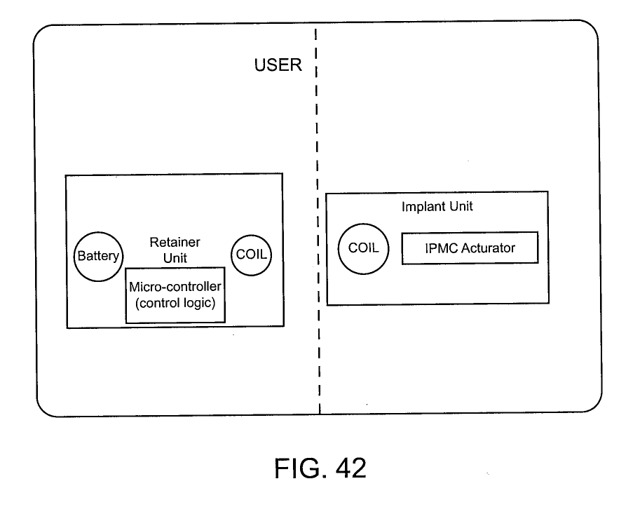

FIG. 42 depicts an embodiment of the invention. The airway implant device comprises of two units—an implant unit and a retainer unit. The implant unit is implanted in a patient and includes an IPMC actuator and a coil. The retainer unit is typically not implanted in the patient and can be worn by the patient prior to going to bed. This unit includes a coil, a battery, and a microcontroller.

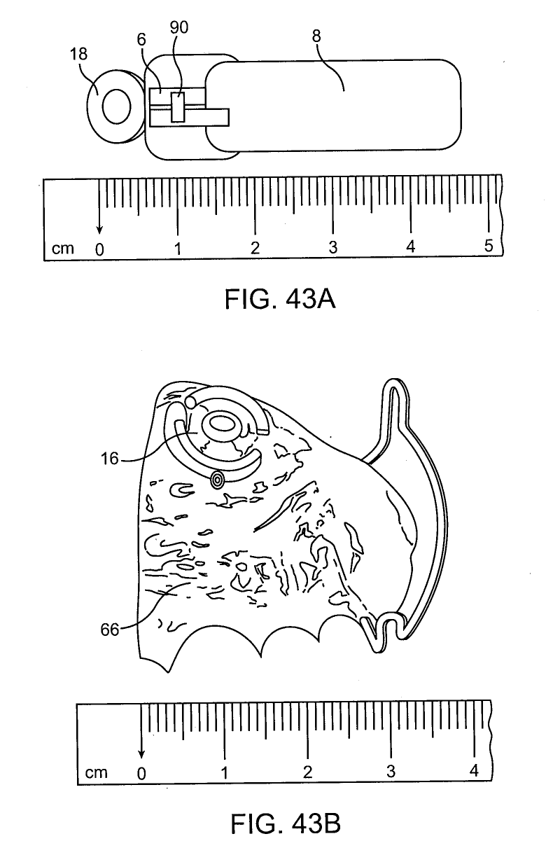

FIG. 43 depicts yet another embodiment of the invention. FIG. 43A is the implant unit, preferably for implantation proximal to or in an airway wall. The implant unit includes an actuator element 8, an inductor 18 in the form of a coil, a controller 90, and connecting elements 6. FIG. 43B depicts the removable retainer with an inductor 16 and a retainer 66.

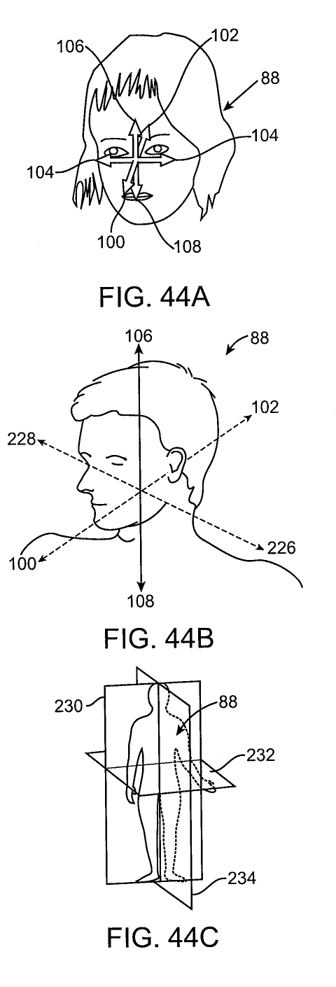

FIGS. 44A, 44B, and 44C illustrate terms used in describing the anatomy of a patient 88 and orientation attributes of the invention. Anterior 100 refers to a part of the body or invention toward the front of the body or invention, or in front of another part of the body or invention. Posterior 102 refers to a part of the invention or body toward the back of the invention or body, or behind another part of the invention or body. Lateral 104 refers to a part of the invention or body to the side of the invention or body, or away from the middle of the invention or body or the middle of the invention or body. Superior 106 refers to a part of the invention or body toward the top of the invention or body. Inferior 108 refers to a part of the invention or body toward the bottom of the invention or body. FIG. 44B illustrates the left 226 and the right 228 sides of a patient anatomy. Various planes of view are illustrated in FIG. 44C, including a coronal plane 230, a transverse plane 232, and a sagittal plane 230.

A preferred embodiment of the device of the present invention comprises an implanted portion 20 comprising an implantable actuator element 8, a housing 112, a first inductor 18, and connecting elements 14 connecting the actuator element 8 to the first inductor 18 within the housing 112; and a non-implanted portion 22 comprising a power source 4 and a second inductor 16 capable of transferring energy to the first inductor 18, wherein the energy of the first inductor 18 energizes the actuator element 8 wherein the actuator element 8 comprises an electroactive polymer element. In a preferred embodiment, the actuator element 8 of the device is implanted in the soft palate 84. The housing 112 of the preferred embodiment is implanted inferior to the hard palate 74. In a preferred embodiment of the device, the housing 112 comprises at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic or thermosetting material.

In a preferred embodiment, the non-implanted portion 22 is in the form of a mouth guard or dental retainer 66. In a preferred embodiment, the non-implanted portion comprises a non-implantable wearable element. In some embodiments, the superior side of the housing 112 comports to the shape of a hard palate 74. In some embodiments, the housing 112 is cast from an impression of a hard palate 74. In still other embodiments, the housing 112 is concave on its superior side. In some embodiments, the housing 112 is convex on its superior side. In some embodiments, the housing 112 comprises bumps 114 on its superior side lateral to a central axis extending from the housing's 112 anterior to its posterior end. In some embodiments, the housing 112 configuration has a substantially smooth rounded superior side. Other configurations for the housing 112 may be contemplated by one having skill in the art without departing from the invention.

In some embodiments, the actuator element 8 is at least partially within the housing 112. In other embodiments, the actuator element 8 is outside the housing 112. The housing 112 is capable of housing and protecting the first inductor 18 and connecting elements 14 between the first inductor 18 and the actuator element 8. In some embodiments, the housing 112 has a roughened surface to increase friction on the housing 112. In some embodiments, the roughened surface is created during casting of the housing 112. In some embodiments, the roughened surface induces fibrosis.

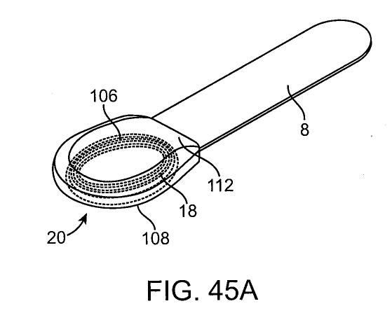

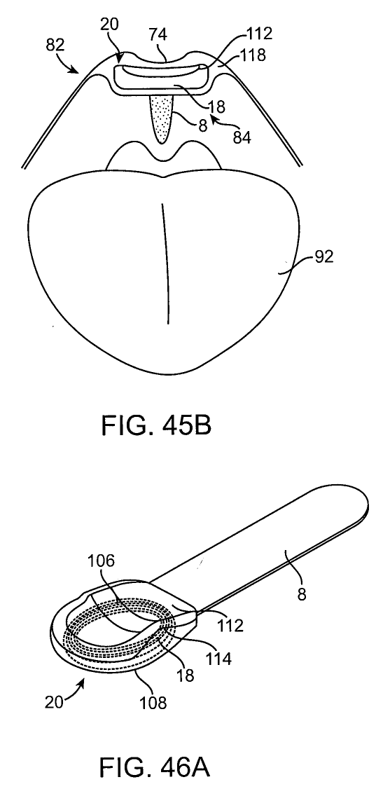

FIG. 45A illustrates one embodiment of the airway implant device comprising a actuator element 8, a first inductor 18, and a housing 112 made from an acrylic and cast with substantially smooth rounded superior and anterior sides. In this embodiment, the actuator element 8 anterior end terminates at about the posterior end of the acrylic housing 112. FIG. 45B illustrates the implant device of FIG. 45A viewed from the anterior side of the implant device, looking toward the posterior end, wherein the implant device is implanted in the palate 116. In the embodiment shown in FIG. 45B, the implant device is implanted such that the housing 112 is in the periosteum 118 inferior to the ridge of the hard palate 74, and the actuator element 8 extends into the soft palate 84.

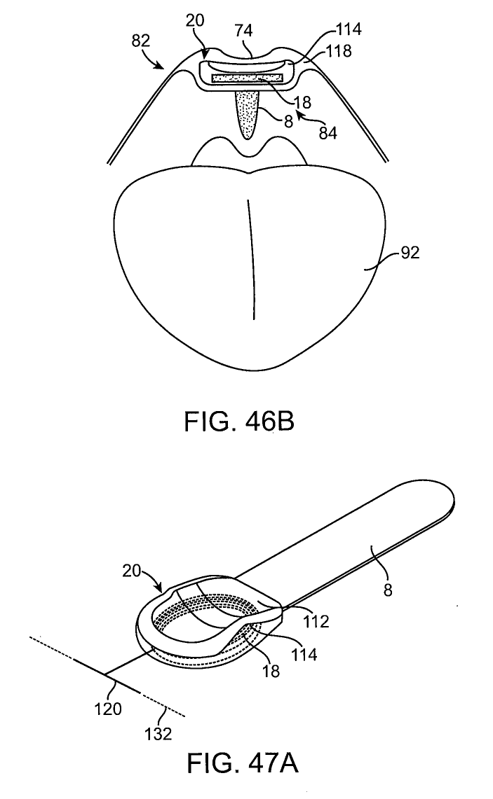

FIG. 46A illustrates an embodiment of the airway implant device that has a actuator element 8, a first inductor 18, and a housing 112 with a smooth rounded inferior side, and at least two bumps 114 on its superior side which, when implanted, comport with the lateral sides of the ridge of the hard palate 74, as shown in FIG. 46B. This configuration reduces rocking of the implant device on the ridge of the hard palate 74 when implanted. In this embodiment, the actuator element 8 anterior end terminates at about the posterior end of the acrylic housing 112. FIG. 46B illustrates the airway implant device of FIG. 46A, viewed from the anterior side of the implant, looking toward the posterior end, wherein the implant device is implanted in the palate 116. In the embodiment shown in FIG. 46B, the implant device is implanted such that the housing 112 is in the periosteum 118 inferior to the ridge of the hard palate 74, and the actuator element 8 extends into the soft palate 84.

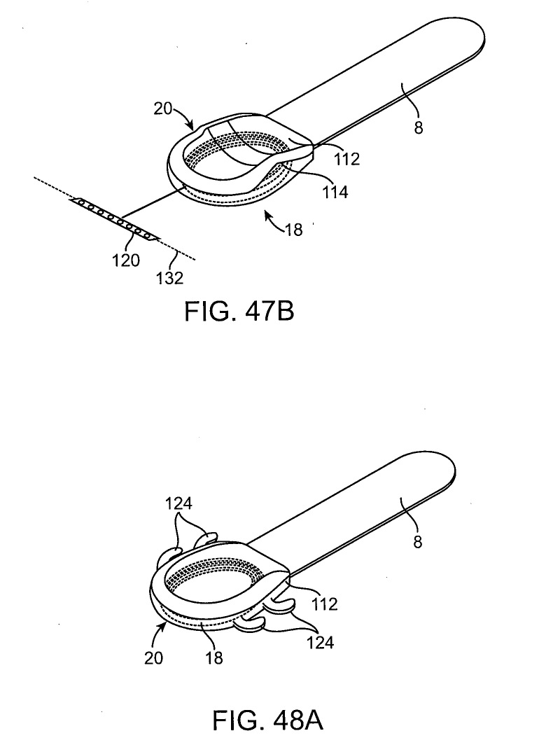

FIG. 47A illustrates an embodiment of the airway implant device having an attachment element 120 at the anterior end of the implant. In this embodiment, the attachment element 120 is T-shaped, however, other configurations and geometries of the attachment element 120 are contemplated in other embodiments, including triangular, circular, L-shaped, Z-shaped, and any geometry within the contemplation of one skilled in the art that would allow attachment of the attachment element to tissue at the anterior end of the implant to fix the position of the implant within the implant cavity.

In some embodiments of the airway implant device having attachment elements 120, the attachment element 120 is a bioabsorbable material. Examples of bioabsorbable materials include, but are not limited to, polylactic acid, polyglycolic acid, poly(dioxanone), Poly(lactide-co-glycolide), polyhydroxybutyrate, polyester, poly(amino acid), poly(trimethylene carbonate) copolymer, poly (ε-caprolactone) homopolymer, poly (ε-caprolactone) copolymer, polyanhydride, polyorthoester, polyphosphazene, and any bioabsorbable polymer.

In another embodiment, the airway implant device comprises an attachment element 120, as shown in FIG. 47B wherein the perforated attachment element 120 comprises at least one hole 122. The hole provides a means for a suture or other attaching device to affix the device to tissue and secure the implant device position. In the case where a suture 132 is used, the suture may or may not be the same suture used by a practitioner to close the original incision made to create a cavity for the implant. The attaching device comprises at least one of a suture, clip, staple, tack, and adhesive.

In some embodiments, the implant may be secured in place, with or without use of an attachment element 120, using an adhesive suitable for tissue, such as cyanoacrylates, and including, but not limited to, 2-octylcyanoacrylate, and N-butyl-2-cyanoacrylate.

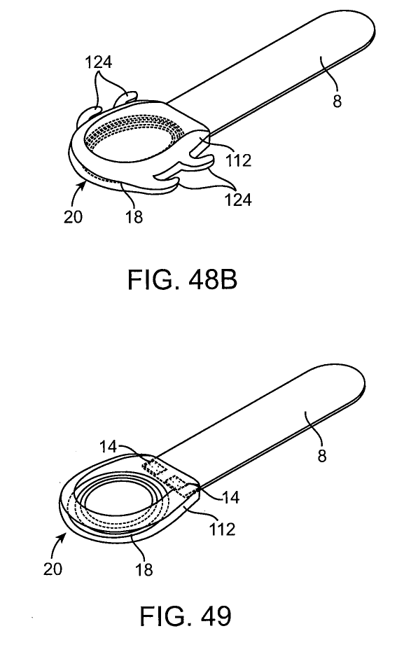

FIG. 48 illustrates an embodiment of the airway implant device wherein the housing 112 has at least one anchor 124. In FIG. 48, the device has four saw-blade like directional anchors 124. The anchors 124 may or may not be made of made of the same materials as the housing 112. Such materials include at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic material. In some embodiments, the device has at least one anchor 124. In some embodiments, the anchor 124 is configured to allow delivery and removal of the implant device with minimal tissue damage. In some embodiments, the anchor 124 is curved. In some embodiments the superior side(s) of the anchor(s) 124 comport with the hard palate 74 surface. In other embodiments, the superior side(s) of the anchor(s) 124 conform to the configuration of the housing 112, options for which are as described elsewhere in this disclosure.

FIG. 49 illustrates a preferred embodiment of the airway implant device wherein the implanted portion 20 comprises power connecting elements 14 comprising a first contact 26 and a second contact 28. In this embodiment, the first contact 26 and second contact 28 have opposing electrical charges, and the housing 112 encases the contacts. In the embodiment shown, the first contact 26 faces in the inferior direction, while the second contact 28 faces in the superior direction. In other embodiments, the first contact 26 faces in the superior direction while the second contact 28 faces in the inferior direction. In some embodiments, the connecting element 14 comprises a non-corrosive conductive material. In some embodiments, the connecting element 14 comprises platinum, gold, silver, stainless steel, or conductive carbon. In some embodiments, the connecting element 14 comprises stainless steel or copper plated with gold, platinum, or silver. In some embodiments, the actuator element 8 stiffens in one direction when a charge is applied to the connecting element 14. In some embodiments, the actuator element 8 deflects when a charge is applied to the connecting element 14.

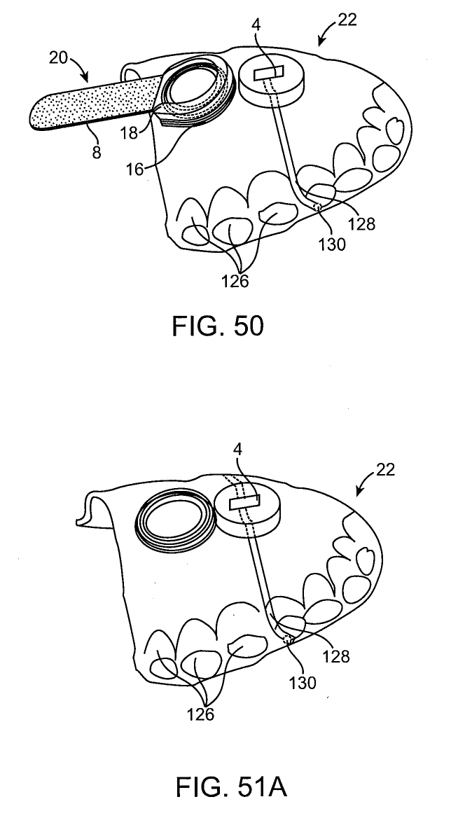

FIG. 50 illustrates an embodiment of the airway implant system wherein the device comprises a non-implanted portion 22 in the form of, and made from similar material as a dental retainer 66. The retainer 66 depicted in FIG. 50 has teeth impressions 126 corresponding to a patient's approximate or exact dentition. Example dental retainer materials include acrylate, polymethylmethacrylate (PMMA), polycarbonate, and nylon. In the embodiment shown in FIG. 50, the non-implanted portion comprises a power source 4 that is rechargeable, a second inductor 16 connected to the power source 4, and ball clamps 128 having two exposed portions 130, said ball clamps 128 connected to the rechargeable power source 4, whereby the exposed portions 130 can recharge the power source 4. The exposed portions 130 are at least partially not covered by retainer material, and are thereby exposed. In the embodiment shown in FIG. 50, the non-implanted portion second inductor 16 transfers energy it receives from the power source 4 to the first inductor 18 of the implanted portion 20, wherein the first inductor 18 energizes the actuator element 8.

In some embodiments, the non-implanted portion 22 does not include ball clamps 128 for recharging the power source 4. In some embodiments, the power source 4 is a rechargeable battery. In some embodiments, the power source 4 is one of a lithium-ion battery, lithium-ion polymer battery, a silver-iodide battery, lead acid battery, a high energy density, or a combination thereof In some embodiments, the power source 4 is removable from the non-implanted portion 22. In some embodiments, the power source 4 is replaceable. In some embodiments, the power source is designed to be replaced or recharged per a specified time interval. In some embodiments, replacing or recharging the power source 4 is necessary no more frequently than once per year. In other embodiments, replacing or recharging the power source 4 is necessary no more frequently than once every six months. In yet other embodiments, replacing or recharging the power source 4 is necessary no more frequently than once or every three months. In yet another embodiment, daily replacing or recharging of the power source is required.

In some embodiments, the power source 4 and second inductor 16 are sealed within the non-implanted portion and the sealing is liquidproof

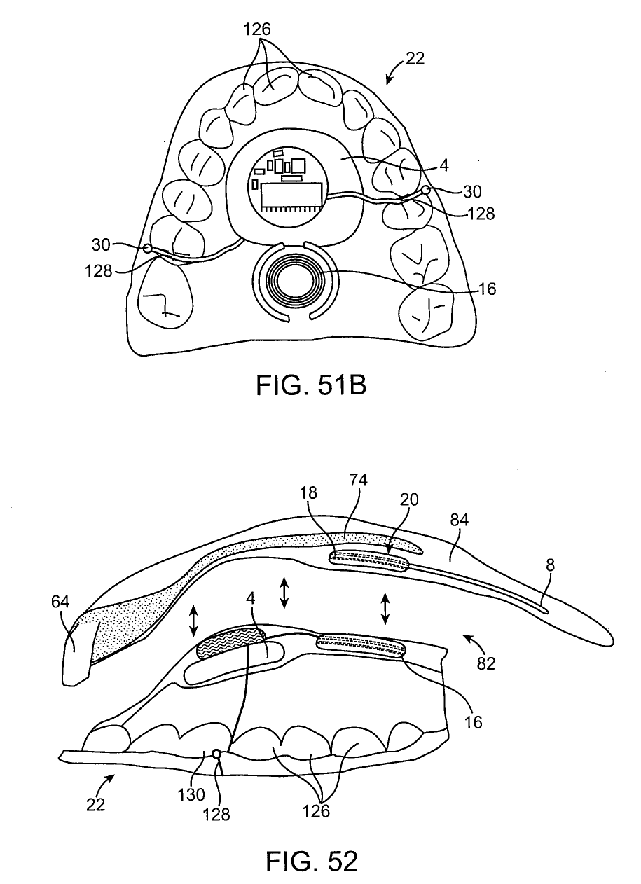

FIGS. 51A, and 51B illustrate different views of an embodiment of the airway implant device non-implanted portion 22 in the form of a retainer 66. In the embodiment depicted, the non-implanted portion 22 comprises a second inductor 16, a power source 4, and at least one balldclamp 128 for recharging the power source 4.

FIG. 52 illustrates an embodiment of the airway implant device implanted in the palate 116. In this embodiment, the housing 112 is implanted inferior to the hard palate 74, whereas the actuator element 8 extends posterior to the housing 112 into the soft palate 84. The non-implanted portion 22 in this embodiment comprises a retainer 66, a power source 4, a second inductor 16, and ball clamps 128 for recharging the power source 4. Other embodiments may comprise none, or some, or all of these elements (the retainer 66, power source 4, second inductor 16, and ball clamps 128), and instead open the airway by means described elsewhere in this specification. In the embodiment depicted in FIG. 52, when the implanted portion 20 of the airway implant device is implanted such that the housing 112 is inferior to the hard palate 74, and when a patient places the retainer 66 in his mouth 82, the retainer 66 having a chargeable second inductor 16 that is positioned within the retainer 66 to align inferior to the implanted first inductor 18, the second inductor 16 transfers energy to the first inductor 18 and the first inductor 18 energizes the actuator element 8. In this embodiment, the actuator element 8 comprises an electroactive polymer (EAP) element, which, when energized by the first inductor 18, opens the airway in which the device is implanted.

### Sensing and Actuation of Airway Implants

One embodiment of the invention is an airway implant device with a sensor for monitoring a condition prior to and/or during the occurrence of an apneic event. Preferably, the sensor monitors for blockage of an airway. The sensor senses the possible occurrence of an apneic event. This sensing of a possible apneic event is typically by sensing a decrease in the airway gap, a change in air pressure in the airway, or a change in air flow in the airway. A progressive decrease in the airway gap triggers the occurrence of an apneic event. Most preferably the sensor senses one or more events prior to the occurrence an apneic event and activates the airway implant to prevent the apneic event. In some embodiments, the airway implant device and the sensor are in the same unit. In other embodiments, the actuator element of the airway implant device is the sensor. In these embodiments, the actuator element acts as both a sensor and actuator. In yet other embodiments, the airway implant device and the sensor are in two or more separate units.

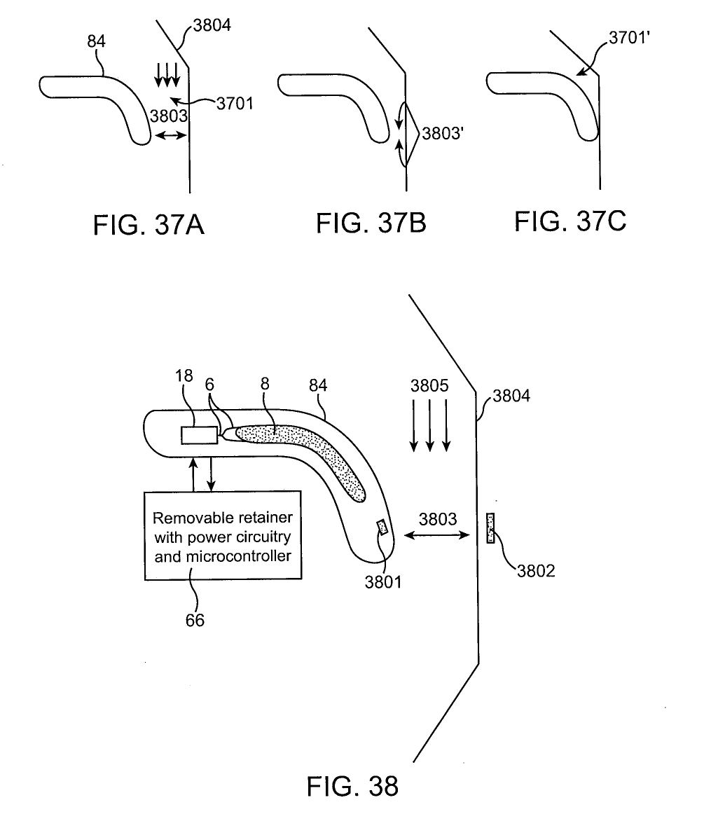

FIG. 37 depicts the occurrence of an apneic event due to the blockage of airway 3701 caused by the movement of the soft palate 84. FIG. 37A shows the soft palate 84 position during normal breathing cycle. An airway gap 3803 is maintained between the soft palate 84 and the laryngeal wall 3804 to maintain airflow 3805. FIG. 37B shows the position of the soft palate 84 just prior to the airway 3701 blockage. It can be seen that the gap 3803′ in this case is smaller than the gap 3803 in FIG. 37A. FIG. 37C shows the soft palate 84 blocking the airway 3701′, leading to the occurrence of an apneic event. In one aspect of the invention, the event shown in FIG. 37C is prevented by taking preemptive action during occurrence of event depicted in FIG. 37B.

One aspect of the invention is an airway implant device with a sensor for sensing the occurrence of apneic events and actuating the device. The invention also includes methods of use of such device.

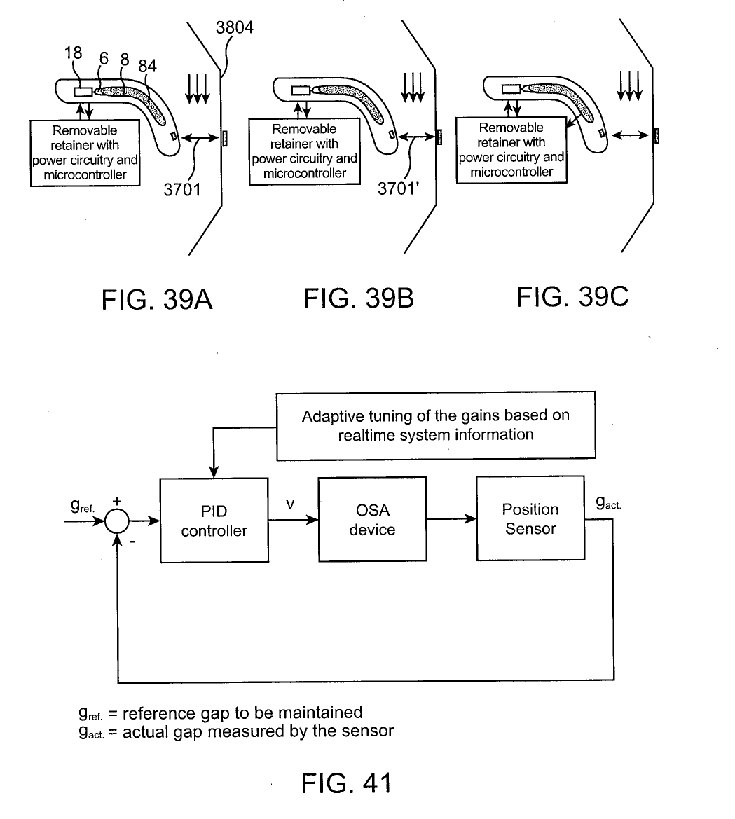

One embodiment of an airway implant device with sensor is depicted in FIG. 38. Non-contact distance sensors 3801 and 3802 are mounted on the laryngeal wall 3804 and also on the soft palate 84 to sense the airway gap between the soft palate 84 and the laryngeal wall 3804. One or more gap values are calibrated into a microcontroller controlling the airway implant device. The functioning of the airway implant device with a sensor is depicted in FIG. 39. During the occurrence of the apneic event the gap between the soft palate 84 and the laryngeal wall 3804 decreases. This gap information is continuously monitored by the airway implant device microcontroller. When the gap becomes smaller than a preset threshold value, the airway implant microcontroller actuates the airway implant, which stiffens the soft palate 84 and the gap between the soft palate 84 and the laryngeal walls 3804 increases. When this gap crosses an upper threshold, the microcontroller powers off the airway implant actuator.

In one embodiment, the operation of the device is as follows:
a) A threshold gap is calibrated into the microcontroller which is present in the removable retainer of the device. This threshold gap corresponds to the gap 3803′ formed by the position of the soft palate with respect to the laryngeal wall as depicted in the FIG. 37B, i.e., a distance at which an apneic event could be triggered or an apneic event occurs. This calibration can take place in real time or when the device is being installed.
b) The non-contact sensor constantly monitors the gap and the information is constantly analyzed by a program present in the microcontroller.
c) The airway implant actuator is in the off state (not powered state) as long as the threshold gap is not reached.
d) When the gap is equal to the threshold gap, the micro controller, powers on the airway implant actuator (on state). This leads to the stiffening of the airway implant actuator, which in-turn stiffens the soft palate.
e) This stiffening of the soft palate prevents the obstruction of the airway and modulates the occurrence of an apneic event.
f) When the gap becomes more than the threshold gap, the micro-controller turns off the airway implant actuator (off state).

Typically, an algorithm in the micro-controller controls the actuation of the actuator. An example of the algorithm is—
if (gap<threshold gap); {Voltage applied to airway implant actuator=high (on state)} or else {Voltage applied to the airway implant actuator=low (off state)}

Complex algorithms, such as adaptive algorithms, can also be used. The objective of the adaptive algorithm can be to selectively control the stiffness of the soft palate by varying the power applied to the airway implant actuator.

Another example of an algorithm to selectively control the stiffness of the soft palate is:
If (gap<or=g)
{Apply full power to the airway implant actuator}
Else
If (gap=g1)
{Voltage applied to airway implant actuator=v1}
Else if (gap=g2)
{Voltage applied to airway implant actuator=v2}
Else if (gap=g3)
{Voltage applied to airway implant actuator=v3}
Note (g1, g2, g3>g)

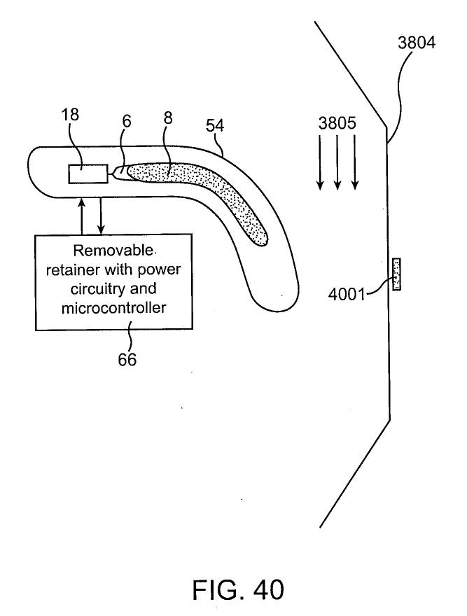

An example of a controller to maintain a predetermined reference gap is shown is FIG. 41. The objective of this algorithm is to maintain an actual airway gap g_{act} as close to the reference airway gap g_{ref} as possible by controlling the airway implant device actuator. The actual airway gap between the soft palate and the laryngeal wall g_{act} is measured and this information is the output of the position sensor. This airway gap information is feedback to the microcontroller which has a controller algorithm embedded in it. In the microcontroller the g_{act} is compared to a g_{ref} and based on the difference between both, the Proportional Integral Derivative (PID) controller generates a controlling voltage which is supplied to the airway implant device. The PID controller can have fixed gains or can have the gains adaptively tuned based on system information.

In alternative embodiments, the sensor can be a wall tension sensor, an air pressure sensor, or an air flow monitoring sensor. In another embodiment, instead of fully turning the airway implant actuator on or off, the actual value of the airway gap can be used to selectively apply varying voltage to the airway implant actuator, hence selectively varying the stiffness of the soft palate. In yet another embodiment, if the airway implant actuator exhibits a lack of force retention over an extended period of time under DC voltage, a feedback control algorithm may be implemented in the microcontroller, which uses the sensory information provided by the sensors to control the stiffness of the soft palate by maintaining the force developed by the airway implant actuator.

Another embodiment of the invention is depicted in FIG. 40. In this embodiment, the wall tension sensed by the wall tension sensor 4001 implanted into the laryngeal wall 3804 is used as a threshold criterion for activating the airway implant actuator. A wall tension sensor can also be placed in a pharyngeal wall or other suitable airway wall. The sensors of this invention can be placed in an airway wall or proximal to an airway wall.

Some of the advantages of the use of an airway sensor with an airway implant device include: optimization of the power consumed by the airway implant device and hence extension of the life of the device; assistance in predicting the occurrence of apneic event, and hence selective activation of the device in order to minimize any patient discomfort; flexibility to use a feedback control system if required to compensate for any actuator irregularities; and possible configuration of the system to interact with an online data management system which will store different parameters related to apneic events for a patient. This system can be accessed by the doctor, other health care providers, and the insurance agency which will help them provide better diagnosis and understanding of the patient's condition.

In preferred embodiments, the airway gap is individually calculated and calibrated for each patient. This information can be stored in the microcontroller. The sensors are described herein mainly in the context of airway implant devices comprising of electroactive polymer actuators. The sensors can also be used with airway implant devices comprising other active actuators, i.e., actuators that can be turned on, off, or otherwise be controlled, such as magnets. The sensors can be used to activate, in-activate, and/or modulate magnets used in airway implant devices. Preferably, the sensors are in the form of a strip, but can be any other suitable shape for implantation. They are typically deployed with a needle with the help of a syringe. The sensor can be made with any suitable material. In preferred embodiments, the sensor is a smart material, such as an IPMC. The sensor is typically in connection with a microcontroller, which is preferably located in the retainer. This connection can be either physical or wireless.

Suitable sensors include, but are not limited to, an electroactive polymer like ionic polymer metal composite (IPMC). Suitable materials for IPMC include perfluorinated polymer such as polytetrafluoroethylene, polyfluorosulfonic acid, perfluorosulfonate, and polyvinylidene fluoride. Other suitable polymers include polyethylene, polypropylene, polystyrene, polyaniline, polyacrylonitrile, cellophane, cellulose, regenerated cellulose, cellulose acetate, polysulfone, polyurethane, polyvinyl acetate. Typically, the electroactive polymer element includes a biocompatible conductive material such as platinum, gold, silver, palladium, copper, and/or carbon. Commercially available materials suitable for use as a sensor include Nafion® (made by DuPont), Flemion® (made by Asahi Glass), Neosepta® (made by Astom Corporation), Ionac® (made by Sybron Chemicals Inc),Excellion™ (made by Electropure). Other materials suitable for use as a sensor include materials with piezoelectric properties like piezoceramics, electrostrictive polymers, conducting polymers, materials which change their resistance in response to applied strain or force (strain gauges) and elastomers.

The airway implant devices of the present invention, with or without the sensor, can be used to treat snoring. For snoring, the sensor can be adapted and configured to monitor air passageways so as to detect the possible occurrence of snoring or to detect the possible worsening of ongoing snoring. Preferably the sensors are capable of detecting relaxation of tissues in the throat, which can cause them to vibrate and obstruct the airway. Other tissues that can be monitored by the sensor include the mouth, the soft palate, the uvula, tonsils, and the tongue.

Another disease that can be treated with the devices of the present invention includes apnea. The sensor preferably monitors the throat tissue for sagging and/or relaxation to prevent the occurrence of an apneic event. Other tissues that can be monitored by the sensor include the mouth, the soft palate, the uvula, tonsils, and the tongue.

### Power Transfer System

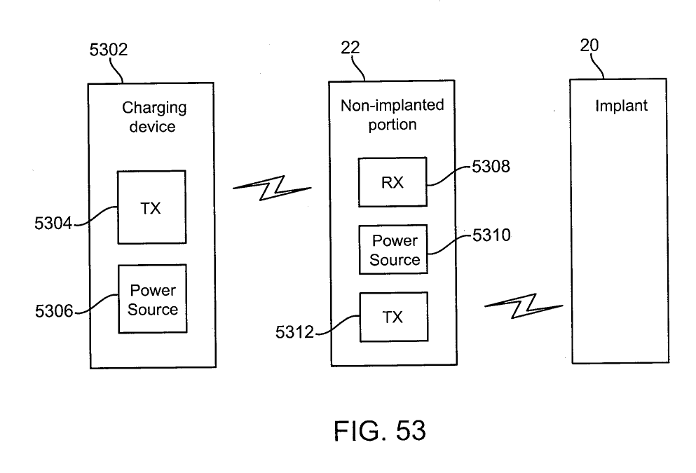

Another aspect of the invention is directed to a wireless power transfer system in which the non-implanted portion receives electrical power from a charging device. FIG. 53 illustrates one embodiment of the wireless power transfer system. As shown, charging device 5302 includes a transmit (TX) circuit 5304 and a power source 5306. Power source 5306 can be a generator or the power grid so that, for example, charger 5302 can plug into a household electrical outlet. Transmit circuit 5304 includes a transducer configured to convert electrical power from power source 5306 to an electromagnetic field suitable for supplying an inductive power transfer to non-implanted portion 22. In some embodiments, transmit circuit 5304 operates at radio-frequencies (RF) and can vary its operating frequency based on power requirements of non-implanted portion 22.

Non-implanted portion 22 includes a receive (RX) circuit 5308, a transmit circuit 5312, and a power source 5310. Receive circuit 5308 includes a second transducer configured to couple with the electromagnetic field from charging device 5302 and to produce a current for powering the operations of non-implanted portion 22. For example, current induced in receive circuit 5308 as part of the inductive transfer can be used to charge power source 5310. Transmit circuit 5312 provides an inductive power transfer to implant 20 as is generally described in connection with FIG. 4. In various embodiments, transmit circuit 5312 includes a third transducer (such as a power transmission coil) configured to provide the inductive power transfer to implant 20. Alternatively, the second transducer can be configured to both receive power from charger 5302 and to supply power to implant 20 depending upon an operating state of non-implanted portion 22.

Advantageously, power source 5310 can be repeatedly charged by receive circuit 5308 and discharged by transmit circuit 5312 in connection with the use of implant 20. Wires and other mechanical connections between non-implanted portion 20 and implant 20 are thereby avoided. In addition, because non-implanted portion 22 has both receive and transmit capabilities, it can communicate with and respond to commands from charging device 5302. In one embodiment, charging device 5302 can collect operating information from non-implanted portion 22 and maintains a log reflecting its usage and condition. Charging device 5302 can, for example, retrieve information about power source 5310 and issue commands to place non-implanted portion 22 into a long-term storage mode in which portions of its transmit and receive circuits 5308, 5310 are deactivated. In some embodiments, charging device 5302 includes a data interface such as a USB connection or serial port through which usage log data is exported for analysis.

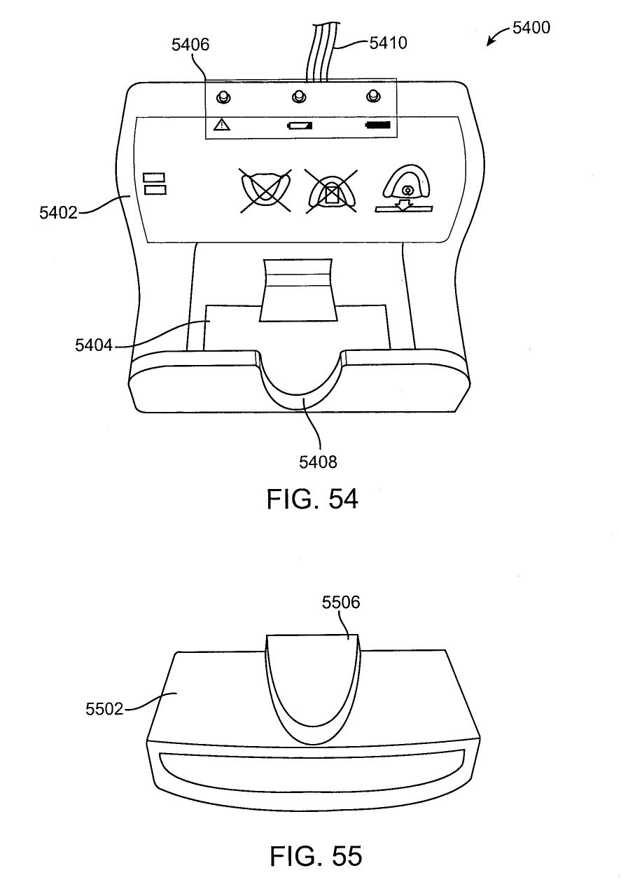

FIG. 54 illustrates one embodiment 5400 of charging device 5302 such as can be used to supply power wirelessly to the non-implanted portion. As shown, housing 5402 of charging device 5400 has a recessed area 5404 adapted to receive the non-implanted portion 22. In this embodiment, the non-implanted portion 22 is a mouthpiece or dental retainer similar to retainer 66 shown in FIGS. 22-23. The retainer is oriented within the recessed area 5404 of the housing 5402 in a manner that promotes effective power transfer. For example, with an inductive power transfer, a pickup coil or inductor attached to the mouthpiece can be aligned with a transmit coil in charging device 5400 to permit effective coupling of an electromagnetic field.

Charging device 5400 also includes status indicators 5406. Status indicators 5406 can be light emitting diodes (LEDs), a liquid crystal display, bar graph elements, icons, or the like, which provide operating information to a user. In this exemplary embodiment, three colored LEDs are used to monitor a power transfer from charging device 5302 to the mouthpiece or retainer such as when charging a battery or other power source. A yellow LED, for example, indicates that power is being transferred from charging device 5400 to the retainer, a green LED signifies that the power transfer is complete, and a red LED alerts the user that a fault condition has been detected.

A timer can be used to control the charging duration and to allow for a cooling period when charging is complete. For example, charging device 5400 may be programmed to transfer power to the retainer for approximately eleven hours and to allow approximately one-half hour for cooling. When the cooling period has expired, the green LED may be illuminated to indicate that the retainer is ready for use. Connector 5410 is also shown on the back side of charging device 5400. Connector 5410 supports communication with electronics disposed within housing 5402 and can be used for calibration and/or to access usage data.

FIG. 55 depicts an exemplary positioning device 5502 such as can be used to align the mouthpiece or retainer with charging device 5400. To facilitate the inductive power transfer, positioning device 5502 is first inserted into recessed area 5404 of housing 5402 by fitting alignment notch 5506 into groove 5408. This creates a space between housing 5402 and positioning device 5502 for holding the mouthpiece or retainer and ensures effective coupling with the electromagnetic field produced by charger 5400. The efficiency of the inductive power transfer is also enhanced. It will be noted, however, that recessed area 5404 and positioning device 5502 are optional. In some embodiments, the mouthpiece or retainer is simply placed for charging onto a flat area of housing 5402 and positioning device 5502 is not used. In each case, power is transferred wirelessly from charging device 5400 to the non-implanted portion 22 where it can be stored and used to power the implant 20.

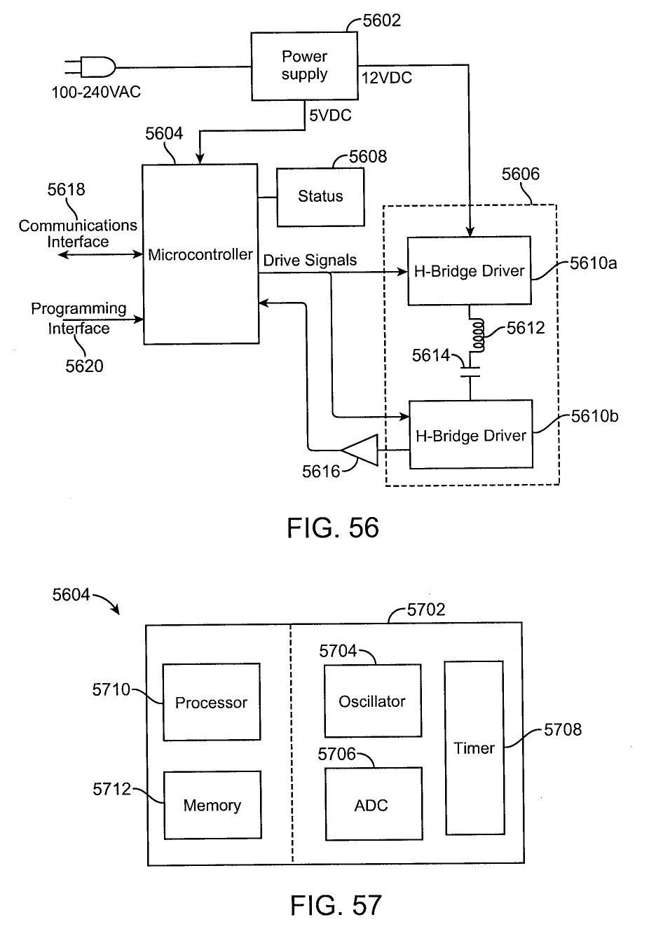

FIG. 56 is a functional block diagram of an exemplary charging device such as charging device 5302. The charging device receives a source voltage of between 100-240 VAC typically from an electrical outlet connected to a power grid or electrical generator. This voltage can optionally be passed through an EMI filter to remove noise components and is then delivered to a power supply 5602. Power supply 5602 produces the supply voltages used by various components of the exemplary charging device. As shown, power supply 5602 delivers a regulated, 5 VDC voltage to microcontroller 5604 and a 12 VDC signal to resonator circuit 5606.

Microcontroller 5604 is the nerve center of the charging device and is configured to control its operations. Microcontroller 5604 supplies drive signals to resonator circuit 5606. In one embodiment, charging device 5302 includes an oscillator (not shown) which produces the drive signals. The oscillator may be calibrated before initial use to produce drive signals at the desired frequency. For example, a frequency of the drive signals can be matched to a resonant frequency of the resonator circuit 5606. The drive signals are applied, under control of the microcontroller 5604, to resonator circuit 5606 to start or stop an inductive power transfer. Alternatively, the oscillator can be embedded within microcontroller 5604 such that its frequency is adjusted internally by microcontroller 5604.

Resonator circuit 5606 receives drive signals from microcontroller 5604 and produces a changing electromagnetic field suitable for inductive power transfer. In one embodiment, resonator circuit 5606 includes an inductor 5612 (also “power transmission coil”), a capacitor 5614, and a pair of H-bridge drivers 5610a, 5610b. Current flowing through the inductor 5612 sets up a magnetic field. As the magnetic field collapses, it charges capacitor 5614. Capacitor 5614 stores the energy in an electric field between its plates. Under the influence of H-bridge drivers 5610, current flows back and forth through inductor 5612 and capacitor 5614 generating an expanding and collapsing electromagnetic field. The electromagnetic field, in turn, supports an inductive power transfer from the charging device to the non-implanted portion of the airway implant system. Although one specific resonator circuit 5606 has been described, many alternatives are possible within the scope of the present invention.

Microcontroller 5604 is also configured to detect a proximity of the non-implanted portion to the charging device. In one embodiment, a current monitor 5616 is coupled to resonator circuit 5606. The current monitor 5616 detects a current level of the resonator circuit 5606 and can include, for example, a resistor shunt amplifier. In operation, as the non-implanted portion 22 is placed near to power transmission coil 5612, it affects the coil's magnetic field and current flow in the power transmission coil changes. Current monitor 5616 detects changes in the current flowing through resonator and provides a signal to microcontroller 5604.

Based on the signal from current monitor 5616, microcontroller 5604 determines the proximity of the non-implanted portion 22 to the charging device. For example, if the signal exceeds a first threshold, microcontroller 5604 determines that the mouthpiece or retainer is present and can be charged. Similarly, if the signal drops below a second threshold, microcontroller 5604 determines that the mouthpiece or retainer has been removed and is no longer charging. This information can be communicated to status indicator 5608 and used to signal an operating state of the charging device. For example, if the current flow exceeds the first threshold, microcontroller 5604 can illuminate a yellow or green LED of status indicator 5608, whereas the LEDs may be extinguished if the current flow is below the second threshold. Microcontroller 5604 also includes a communications interface 5618 and a programming interface 5620.

FIG. 57 depicts an exemplary microcontroller 5604 such as can be used with charging device 5302. As shown, microcontroller 5604 includes embedded peripherals 5702 as well as processor 5710 and memory 5712 elements. Embedded peripherals 5702 include oscillator 5704, analog-to-digital converter (ADC) 5706, and timer 5708. Oscillator 5704 generates programmable drive signals having a frequency that is determined by processor 5710. The drive signals are supplied, for example, to a resonator circuit 5606 for controlling power transferred from the charging device. Analog-to-digital converter 5706 produces a digital signal corresponding to the output of current monitor 5616 which can then be compared by processor 5710 to one or more threshold values to determine a proximity of the non-implanted portion 22. Timer 5708 is programmable by processor 5710 and can be used, among other things, to determine charging and cooling intervals. In some embodiments, microcontroller 5604 also includes a real-time clock (RTC) used in connection with event logging as described herein.

Memory 5712 can include read-only memory (ROM) and random-access memory (RAM). In one embodiment, memory 5712 stores programming instructions as well as calibration and usage data. Programming instructions can be loaded through programming interface 5620 and typically include software for controlling operation of the charging device and for communicating with other devices. For example, programming instructions can provide a command interface for sending usage data and for receiving calibration data through communication port 5618. Also, programming instructions can support a user interface provided by status indicators 5608.

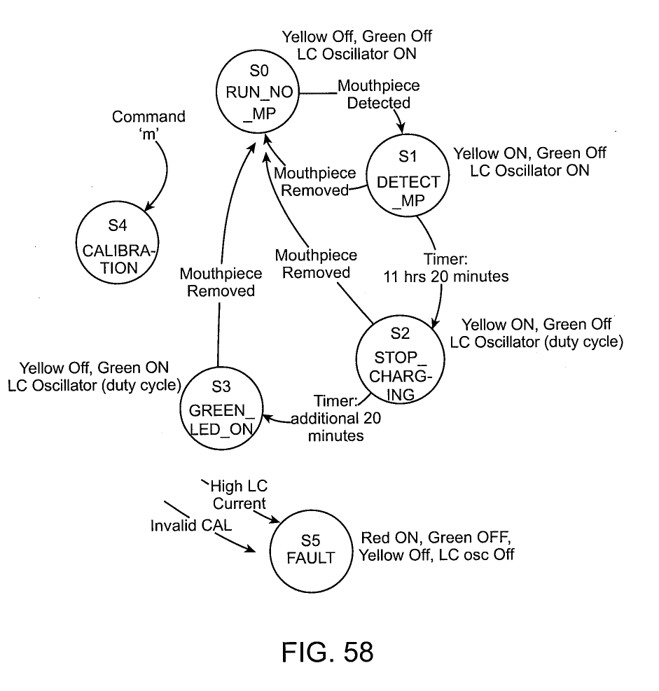

FIG. 58 is a state diagram showing transitions between exemplary operating states of processor 5710. A state machine for performing these functions may be implemented through a combination of programming instructions, embedded peripherals, and other hardware elements as previously discussed. In first state (S0), the charging device is operating and the non-implanted portion is not detected. The resonator circuit is active and the external device 5302 is therefore ready to transfer power. This state is signified by, for example, turning off the LED status indicators.

When the non-implanted portion (mouthpiece) is detected, processor 5710 transitions to state S1. This can occur, for example, when inductive power transfer begins and may be detected by a change in current through the power transmission coil. In response, processor 5710 starts a charging timer and illuminates a yellow LED to indicate that the charging device is supplying power to the mouthpiece. In an exemplary embodiment, the timer expires after approximately 11 hours and 20 minutes. Generally speaking, the timer interval corresponds to the charge rate and can vary based upon operational requirements. For example, relatively low charging rates may be preferred to reduce heat and to facilitate the use of low power components in the non-implanted portion, whereas higher charging rates can be utilized if more frequent use of the non-implanted portion is desired.

Upon expiration of the charging timer, processor 5710 transitions to state S2 in which the resonator circuit enters an idle state. During the idle state, for example, processor 5710 periodically activates the resonator circuit to detect the proximity of the mouthpiece. In some embodiments, this periodic checking also serves to reset a timer in the mouthpiece which controls power transfer to the implant. For example, periodic proximity detection by the charging device can reset the timer of a fully-charged mouthpiece such that the mouthpiece does not begin sourcing an inductive power transfer before it is removed from the charger. In state S2, processor 5710 also activates a cooling timer to permit the mouthpiece to cool for a predetermined interval (such as 20 minutes) following charging. The yellow LED remains illuminated during the cooling interval.

When the cooling timer expires, processor 5710 transitions to state S3 which signifies that charging (and cooling) is complete. At this point, the green LED is illuminated and the resonator circuit remains in the idle state. If the mouthpiece is removed at any point during states S1-S3, processor 5710 reverts to state S0 until the mouthpiece is again detected. Also, processor 5710 transitions to state S4 in response to external commands. For example, if the command ‘m’ is received through communications interface 5618, processor 5710 enters a calibration mode. In calibration mode, operational settings such as the drive signal frequency, threshold values for proximity detection, and current date/time information may be entered and stored in memory 5712. If at any point during operation, excessive current levels or bad calibration data is detected, processor 5710 transitions to state S5. In state S5, a fault is indicated by illuminating the red LED and the resonator circuit is disabled.

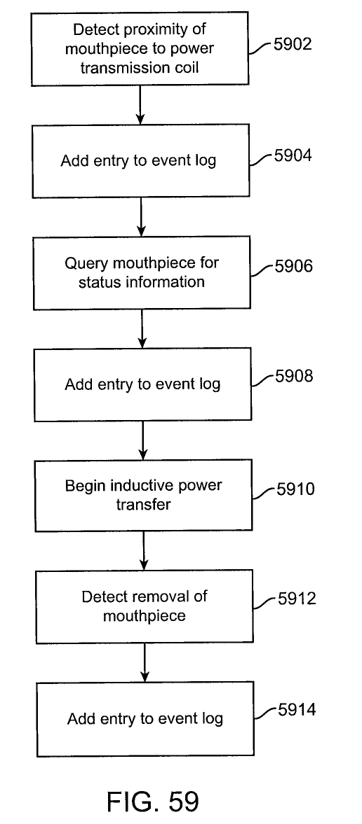

FIG. 59 is a flowchart illustrating exemplary processing operations. The steps depicted in FIG. 59 can be performed by processor 5710 or, more generally, by microcontroller 5604. In a first step 5902, the proximity of the mouthpiece to the charging device is detected. This can occur when the mouthpiece is placed on or near the charger and, for example, can also trigger a transition in processor 5710 from state S0 to S1 as previously discussed.

At step 5904, an entry is added to the event log. In some embodiments, event data is stored in a memory or non-volatile storage device from which it can later be retrieved for analysis. For example, the frequency with which the non-implanted portion 22 is used can be estimated based on the number and frequency of charging events. Similarly, the duration of each use can be estimated based upon the duration of each charging event. Typically, events stored in the event log are time stamped such as with the output of a real-time clock. Thus, when the presence of the mouthpiece is detected, an arrival event stamped with the current time is added to the event log.

After logging the arrival event, the charging device queries the mouthpiece or retainer 5906 for status information. In one embodiment, the charging device communicates with the retainer or mouthpiece using a type of on-off keying. For example, when processor 5710 detects that the mouthpiece is near to the power transmission coil, it toggles the resonator circuit on and off at predetermined intervals representing a status-query command. The processor 5710 then waits for a response to the query and, at step 5908, records status information received from the mouthpiece portion in the event log. By way of illustration, the mouthpiece may transmit a voltage level of its internal battery to the charging device. The voltage level can be compared to the rate at which a mouthpiece battery is discharged during normal operation and can thus provide a usage estimate. Similarly, abnormal voltage levels can provide an alert that the mouthpiece may need service.

When communication with the mouthpiece is complete, at step 5910, inductive power transfer begins. At step 5912, removal of the mouthpiece from the charging device is detected. This can occur, for example, when processor 5710 transitions from state S2 to state S3 or when the mouthpiece is removed before charging is complete. In one embodiment, the charging device periodically queries the mouthpiece for status information and stops charging when it is determined that the mouthpiece battery is fully charged. At step 5914, an entry in the event log is added when charging is complete or the mouthpiece has been removed. Final status information, if available, may also be added to the event log.

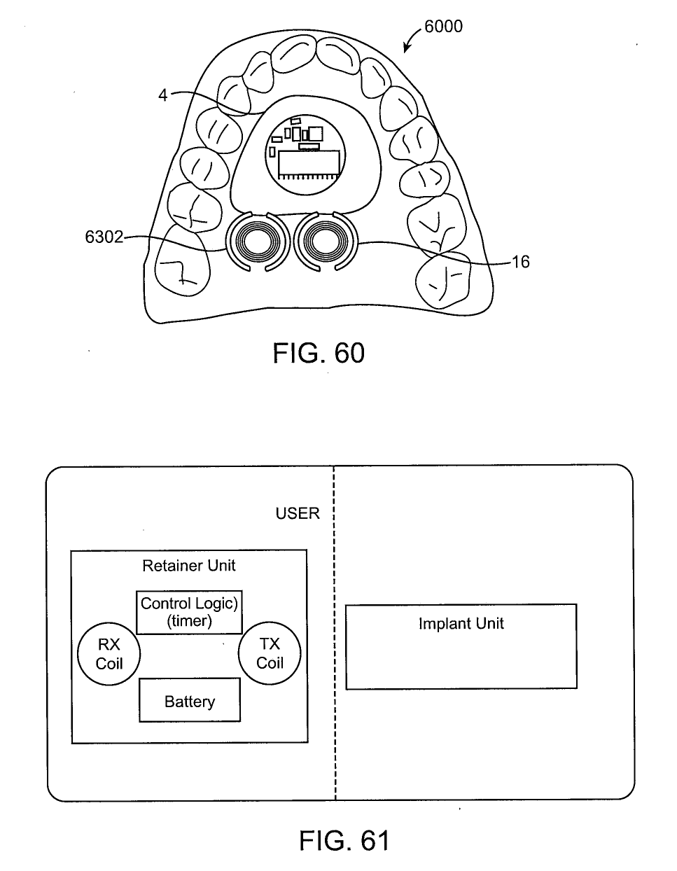

FIG. 60 illustrates an embodiment of the non-implanted portion 6000 such as can be used with the wireless power transfer system of FIG. 53. As shown, the non-implanted portion is a retainer 6000 similar to that described in connection with FIG. 51B. However, it will be noted that retainer 6000 has no ball clamps or other mechanical connections for transferring power to power source 4. Instead, retainer 6000 is configured to receive an inductive power transfer from charging device 5302 and to source power wirelessly to implant 20. In operation, third inductor 6302 couples with charging device 5302 and receives an inductive power transfer. Power from charging device 5302 is delivered to power source 4 which can, for example, be a lithium polymer battery or other rechargeable power cell. Second inductor 16 transfers power from power source 4 to first inductor 18 as described herein.

FIG. 61 depicts a further embodiment of the invention. Similar to FIG. 42, the airway implant device comprises an implant unit and a retainer unit. The retainer unit includes a receive (RX) coil and a transmit (TX) coil. During charging, the RX coil is inductively coupled to the power transmission coil of a charging device and supplies a charging current to the battery. The charging current also resets a timer which forms part of a control logic block. When charging is interrupted, the timer is activated. After a predetermined time, the control logic causes the battery to power the TX coil for transferring power to the implant unit.

In an alternative embodiment, the separate RX and TX coils shown in FIG. 61 are replaced by a single RX/TX coil. The operation of the RX/TX coil is determined by a mode-select portion of the control logic block. In charging mode, the RX/TX coil supplies a charging current to the battery. In transmit mode, the RX/TX coil receives power from the battery. As with the two-coil embodiment of FIG. 61, a timer may be used to control mode selection. For example, the timer can include an RC circuit configured so that a capacitor voltage controls the timing of the inductive power transfer to the implant unit.

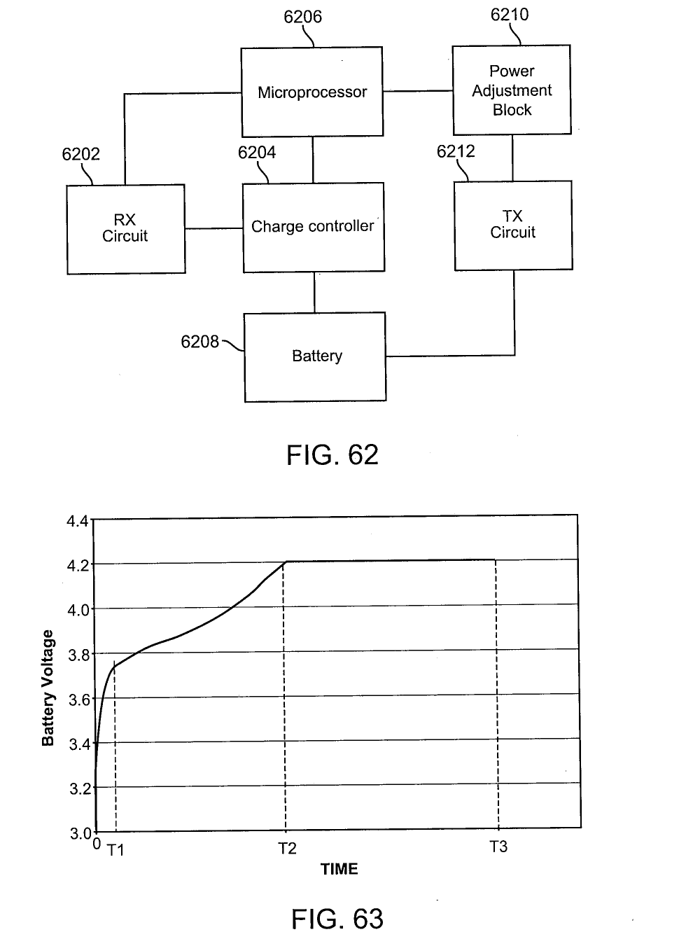

FIG. 62 is a functional block diagram showing electronics included with one embodiment of the non-implanted portion. Receive (RX) circuit 6202 is configured to receive an inductive power transfer from an external source such as charging device 5302. In some embodiments, RX circuit 6202 includes a pickup coil in which a current flows by electromagnetic induction when the non-implanted portion is positioned on or around the external device. RX circuit 6202 can also include a full-wave rectifier and low-pass filter for producing a DC voltage from the induced current.

RX circuit 6202 is coupled to charge controller 6204 and microprocessor 6206. Charge controller 6204 receives the DC voltage from RX circuit 6202 and delivers a charging current to battery 6208. Charge controller 6204 also monitors the health of battery 6208 and provides status information to microprocessor 6206. For example, charge controller 6204 can protect battery 6208 from over-voltage conditions by limiting the voltage at its output to a predetermined level. Charge controller 6204 can also protect against battery failure or low-voltage conditions by providing a trickle-charge in which current flow is greatly reduced. Additionally, charge controller 6204 can monitor and report the temperature of battery 6208 to microprocessor 6206.

In one embodiment, charge controller 6204 is configured to provide a constant-current (CC), constant-voltage (CV) charge to battery 6208. For example, battery 6208 can be a lithium polymer cell with an operational range of about 3.0-4.2 volts. During a constant-current portion of the charge cycle, current is delivered to battery 6208 at a more or less constant rate thereby increasing the voltage across its terminals. CC charging is shown in FIG. 63 by the interval from T1 to T2. When the target voltage is reached, charge controller 6204 switches to CV mode and maintains its output at a constant voltage. This is illustrated in FIG. 63 by the interval from T2 to T3.

Microprocessor 6206 controls the operation of the non-implanted portion and can included embedded peripherals similar to those discussed with microcontroller 5604. When charging battery 6208, microprocessor 6206 can monitor the status information received from charger controller 6204 and can deactivate parts of the non-implanted portion if problems are detected. For example, microprocessor 6206 can monitor current levels through a pickup coil in RX circuit 6202 and can decouple charge controller 6204 from the RX circuit 6202 if an excessive current is detected. Also, microprocessor 6206 can halt charging based upon a voltage level or temperature of battery 6208 and can deenergize TX circuit 6212 if it is determined that battery 6208 can no longer support a minimum transmit power.

Microprocessor 6206 also controls TX circuit 6212 and can communicate status information. Status information, for example, can include data such as a charging voltage or current levels in addition to a voltage of battery 6208 and current transmit power settings of the non-implanted portion. In one embodiment, microcontroller 6206 detects a command from an external device (such as charging device 5302) and responds by transmitting one or more pieces of status information. Commands from the external device can also be used to place the non-implanted portion into a long-term storage mode.

Commands from the external device and responses to such commands may be communicated via TX circuit 6212 using frequency modulation, amplitude modulation, on-off keying, and other techniques as known in the relevant art. In this way, battery 6208 is wirelessly charged and the non-implanted portion provides status information which can be logged and analyzed as described herein. Microprocessor 6206 can also provide a timer function as discussed in connection with FIG. 61 through which TX circuit 6212 is activated at a predetermined time after battery charging has completed.

Power adjustment block 6210 cooperates with microprocessor 6206 to control power transferred to the implant. In one embodiment, power adjustment block 6210 includes an RDAC (digital potentiometer) the setting of which is varied by microprocessor 6206 to maintain a steady voltage at the implant device. For example, an offset can exist between the mouthpiece or retainer and the implant. During calibration, an initial RDAC value can be established so that, for example, sufficient power is transferred from TX circuit 6212 to support operation of the implant. In one exemplary embodiment, calibration is based upon a separation distance of approximately 2 mm and the RDAC setting is chosen to support a target voltage of about 1.9V at the implant.

The offset can vary somewhat relative to the value establishing during calibration. In particular, the distance between a power transmission coil of TX circuit 6212 and a pickup coil of the implant may differ from calibration estimates. To compensate for such differences, microprocessor 6206 measures a current flow in TX circuit 6212 and delivers a control signal to power adjustment block 6210. The measured current, for example, provides an indication of the power supplied to the implant.

In one embodiment, power adjustment block 6210 includes a monitor circuit such as current monitor 5616 and operates to maintain a steady voltage at the implant independent of the battery 6208 voltage. In a manner similar to that described in FIG. 56, microprocessor 6206 converts the output of the current monitor into a digital value and determines whether to adjust the level of TX circuit 6212. If an adjustment is needed, in one embodiment, microprocessor 6206 obtains a new RDAC setting by accessing a lookup table based on the digital value. The lookup table, for example, can provide RDAC settings with which to adjust the output of TX circuit 6212 so that sufficient operating power is maintained at the implant.

TX circuit 6212 receives power from battery 6208 and provides an inductive power transfer to the implant. In one embodiment, TX circuit 6212 includes a resonator circuit similar to resonator 5606. Specifically, transmit circuit 6212 can include a pair of H-bridge drivers, a power transmission coil, and a capacitor configured to resonate and to support an inductive coupling with the implant. Microprocessor 6206 activates and deactivates TX circuit 6212 and controls its output via power adjustment block 6210. Although described separately, it will be recognized that the functions of TX circuit 6212 and power adjustment block 6210 may be combined and that both may be supported by peripherals such as an oscillator, analog-to-digital converter, and current sensor, either embedded in or separate from microprocessor 6206.

The implants described herein are preferably implanted with a deployment tool. Typically, the implantation involves an incision, surgical cavitation, and/or affixing the implant.

### Methods of Making Electroactive Polymer Element

In some embodiments, the EAP element is an IPMC strip which is made from a base material of an ionomer sheet, film or membrane. The ionomer sheet is formed using ionomer dispersion.

IPMC is made from the base ionomer of, for example, polyethylene, polystyrene, polytetrafluoroethylene, polyvinylidene fluoride (PVDF) (e.g., KYNAR® and KYNAR Flex®, from ATOFINA, Paris, France, and SOLEF®, from Solvay Solexis S. A., Brussels, Belgium), hydrophilic-PVDF (h-PVDF), polyfluorosulfonic acid based membranes like NAFION® (from E.I. Du Point de Nemours and Company, Wilmington, Del.), polyaniline, polyacrylonitrile, cellulose, cellulose acetates, regenerated cellulose, polysulfone, polyurethane, and combinations thereof. The conductive material that is deposited on the ionomer can be gold, platinum, silver, palladium, copper, graphite, conductive carbon, or combinations thereof. Conductive material is deposited on the ionomer either by electrolysis process, vapor deposition, sputtering, electroplating, or combination of processes.

The IPMC is cut into the desired implant shape for the EAP element. The electrical contact (e.g., anode and cathode wires for EAP element) is connected to the IPMC surfaces by, for example, soldering, welding, brazing, potting using conductive adhesives, or combinations thereof. The EAP element is configured, if necessary, into specific curved shapes using mold and heat setting processes.

In some embodiments, the EAP element is insulated with electrical insulation coatings. Also, the EAP element can be insulated with coatings that promote cell growth and minimize fibrosis, stop cell growth, or kill nearby cells. The insulation can be a biocompatible material. The EAP element is coated with polymers such as polypropylene, poly-L-lysine, poly-D-lysine, polyethylene glycol, polyvinyl alcohol, polyvinyl acetate, polymethyl methacrylate, or combinations thereof. The EAP element can also be coated with hyaluronic acid. The coating is applied to the device by standard coating techniques like spraying, electrostatic spraying, brushing, vapor deposition, dipping, etc.

In one example, a perfluorosulfonate ionomer, PVDF or h-PVDF sheet is prepared for manufacturing the EAP element. In an optional step, the sheet is roughened on both sides using, for example, about 320 grit sand paper and then about 600 grit sand paper; then rinsed with deionized water; then submerged in isopropyl alcohol (IPA); subjected to an ultrasonic bath for about 10 minutes; and then the sheet is rinsed with deionized water. The sheet is boiled for about 30 minutes in hydrochloric acid (HCL). The sheet is rinsed and then boiled in deionized water for about 30 minutes. The sheet is then subject to ion-exchange (i.e., absorption). The sheet is submerged into, or otherwise exposed to, a metal salt solution at room temperature for more than about three hours. Examples of the metal salt solution are tetraammineplatinum chloride solution, silver chloride solution, hydrogen tetrachloroaurate, tetraamminepalladium chloride monohydrate or other platinum, gold, silver, carbon, copper, or palladium salts in solution. The metal salt solution typically has a concentration of greater than or equal to about 200 mg/100 ml water. 5% ammonium hydroxide solution is added at a ratio of 2.5 ml/100 ml to the tetraammineplatinum chloride solution to neutralize the solution. The sheet is then rinsed with deionized water. Primary plating is then applied to the sheet. The sheet is submerged in water at about 40° C. 5% solution by weight of sodium borohydride and deionized water is added to the water submerging the sheet at 2 ml/180 ml of water. The solution is stirred for 30 minutes at 40° C. The sodium borohydride solution is then added to the water at 2 ml/180 ml of water and the solution is stirred for 30 minutes at 40° C. This sodium borohydride adding and solution stirring is performed six times total. The water temperature is then gradually raised to 60° C. 20 ml of the sodium borohydride solution is then added to the water. The solution is stirred for about 90 minutes. The sheet is then rinsed with deionized water, submerged into 0.1N HCl for an hour, and then rinsed with deionized water.

In some embodiments, the sheet receives second plating. The sheet is submerged or otherwise exposed to a tetraammineplatinum chloride solution at a concentration of about 50 mg/100 ml deionized water. 5% ammonium hydroxide solution is added at a rate of 2 ml/100 ml of tetrammineplatinum chloride solution. 5% by volume solution of hydroxylamine hydrochloride in deionized water is added to the tetraammineplantium chloride solution at a ratio of 0.1 of the volume of the tetraammineplatinum chloride solution. 20% by volume solution of hydrazine monohydrate in deionized water is added to the tetraammineplatinum chloride solution at a ratio of 0.05 of the volume of the tetraammineplantinum chloride solution. The temperature is then set to about 40° C. and the solution is stirred.

A 5% solution of hydroxylamine hydrochloride is then added at a ratio of 2.5 ml/100 ml of tetraammineplatinum chloride solution. A 20% solution of hydrazine monohydrate solution is then added at a ratio of 1.25 ml/100 ml tetraammineplatinum chloride solution. The solution is stirred for 30 minutes and the temperature set to 60° C. The above steps in this paragraph can be repeated three additional times. The sheet is then rinsed with deionized water, boiled in HCl for 10 minutes, rinsed with deionized water and dried.

In some embodiments, the polymer base is dissolved in solvents, for example dimethyl acetamide, acetone, methylethyle ketone, toluene, dimethyl carbonate, diethyl carbonate, and combinations thereof. The solvent is then allowed to dry, producing a thin film. While the solution is wet, a low friction, (e.g., glass, Teflon) plate is dipped into the solution and removed. The coating on the plate dries, creating a think film. The plate is repeatedly dipped into the solution to increase the thickness of the film.

Polyvinyl alcohol, polyvinyl pyrrolidone, polyinyl acetate or combinations thereof can be added to a PVDF solution before drying, thus contributing hydrophilic properties to PVDF and can improve ion migration through the polymer film during manufacture. Dye or other color pigments can be added to the polymer solution.

### Method of Using

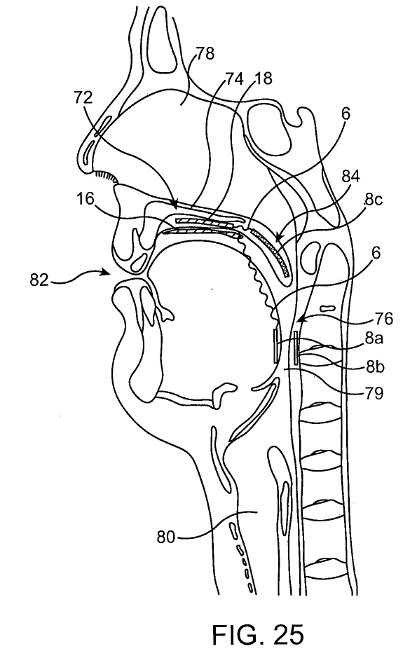

FIG. 25 illustrates an embodiment of a method of the airway implant device of the present invention. In this embodiment, the first inductor 18 is implanted in the mouth roof 72, for example in or adjacent to the hard palate 74. Wire leads 6 connect the first inductor 18 to the actuator elements 8a, 8b, and 8c. A first actuator element 8a is implanted in the base of the tongue at the pharynx wall 76. A second actuator element 8b is integral with the first actuator element 8a (e.g., as two sections of a hollow cylindrical actuator element 8, such as shown in FIG. 17). The first and second actuator elements 8a and 8b can be separate and unattached elements. The third actuator element 8c is implanted in the uvula and/or soft palate 84. The actuator elements 8 can also be implanted in the wall of the nasal passages 78, higher or lower in the pharynx 79, such as in the nasal pharynx, in the wall of the trachea 80, in the larynx (not shown), in any other airway, or combinations thereof. The second inductor 16 is worn by the patient in the mouth 82. The second inductor 16 is connected to an integral or non-integral power source. The second inductor 16 comprises one or multiple induction coils. The second inductor 16 inductively transmits RF energy to the first inductor 18. The first inductor 18 changes the RF energy into electricity. The first inductor 18 sends a charge or current along the wire leads 6 to the actuator elements 8a, 8b, and 8c. The actuator elements 8a, 8b, and 8c are energized by the charge or current. The energized actuator elements 8a, 8b, and 8c increase the stiffness and/or alter the shape of the airways. The energized actuator elements 8a, 8b, and 8c modulate the opening of the airways around which the actuator elements 8a, 8b, and 8c are implanted. The non-energized actuator elements 8a, 8b, and 8c are configured to conform to the airway around which the actuator elements 8a, 8b, and 8c are implanted. The non-energized actuator elements 8a, 8b, and 8c are flexible and soft.

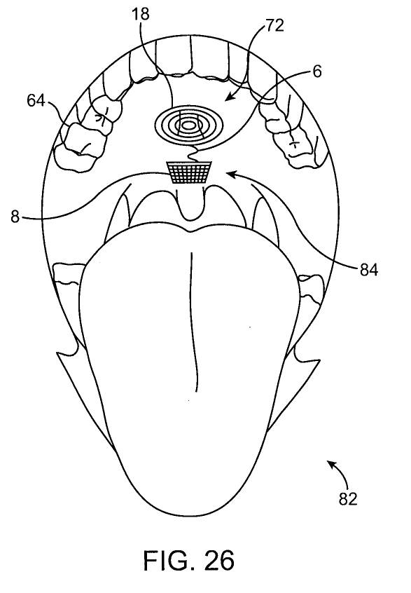

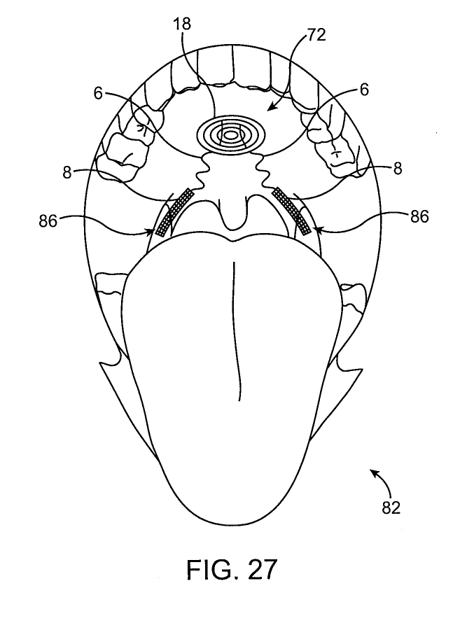

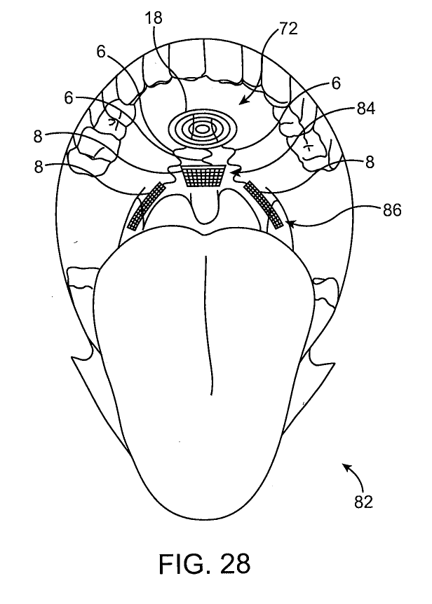

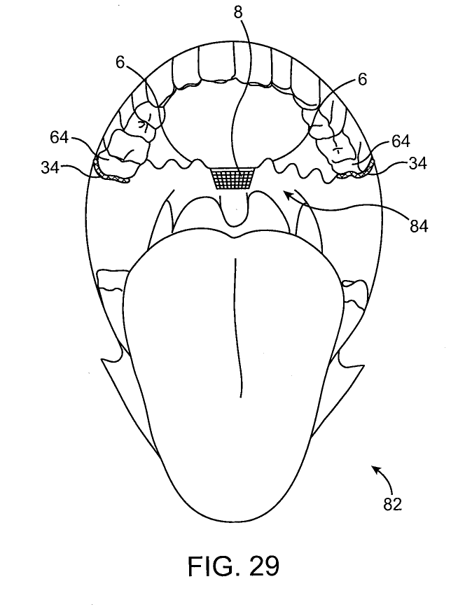

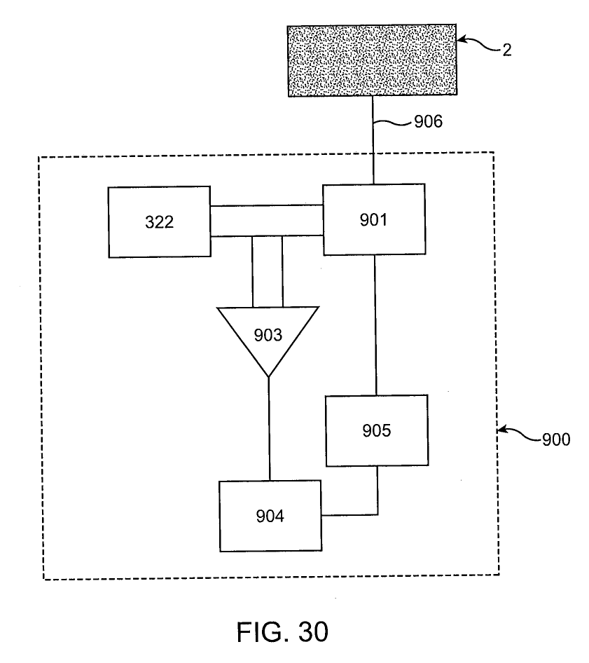

FIG. 26 illustrates another embodiment of the invention. In this embodiment, the first inductor 18 is implanted in the mouth roof 72 and attached to a actuator element 8 via the wire lead 6. The actuator element 8 is preferably in the soft palate 84. In another embodiment, FIG. 27 illustrates that the first inductor 18 is implanted in the mouth roof 72 and attached to two actuator elements 8 via two wire leads 6. The actuator elements 8 are implanted in side walls 86 of the mouth 82. In yet another embodiment, as illustrated in FIG. 28, the first inductor 18 is implanted in the mouth roof 72 and attached to three actuator elements 8 via three wire leads 6. The actuator elements 8 are implanted in the soft palate 84 and the side walls 86 of the mouth 82. FIG. 29 illustrates an embodiment in which the first conductors (not shown, e.g., the tooth sockets), are attached to, and in conductive electrical communication with, the second conductors. The retainer 66, such as shown in FIG. 23, can be worn by the patient to energize the actuator element 8. The tooth sockets are removably attached to the first conductors 34. The first conductors 34 are dental fillings, conductive posts adjacent to and/or through the teeth 64.

FIG. 33 illustrates an embodiment in which a patient 88 has the first transducer (not shown) implanted in the patient's cheek and wears the non-implanted portion 22, such as shown in FIG. 24, on the outside of the patient's cheek. The non-implanted portion 22 energizes the implanted portion (not shown).

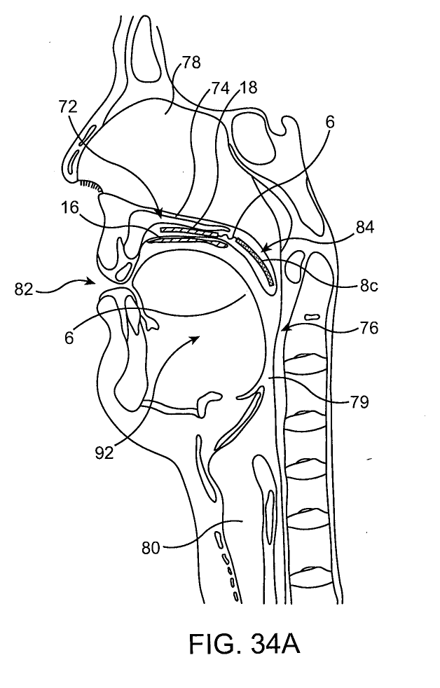

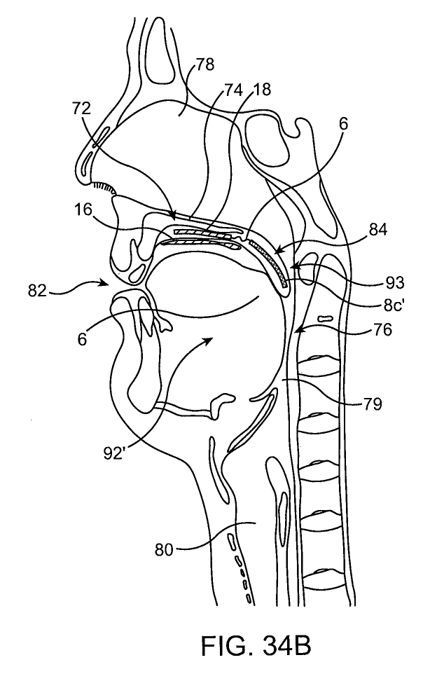

FIGS. 34-36 depict some of the ways in which the implant devices function to open the airways. FIG. 34A and 34B depict a side view of a patient with a soft palate implant 8c and a non-implanted portion of the device, with a second inductor 16, which in this case is a wearable mouth piece. The wearable mouth piece includes a transmitter coil, a power source, and other electronics, which are not depicted. Also, shown is a first inductor 18. The implant device has the ability to sense and deflect the tongue so as to open the airway. FIG. 34A depicts the tongue 92 in its normal state. During sleep, when the tongue collapses 92′, as shown in FIG. 34B, the actuator element 8c′ senses the collapsed tongue and is energized via the mouthpiece and first inductor and it stiffens to push away the tongue from the airway and keeps the airway open. This opening of the airway can be partial or complete. In some embodiments, particularly the embodiments without the sensor, the implant is powered when the patient is asleep such that the actuator element 8 is energized and keeps the collapsed tongue away from the airway.

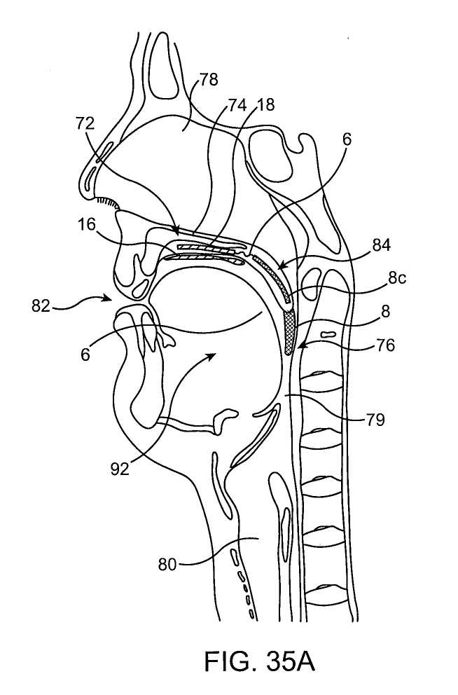

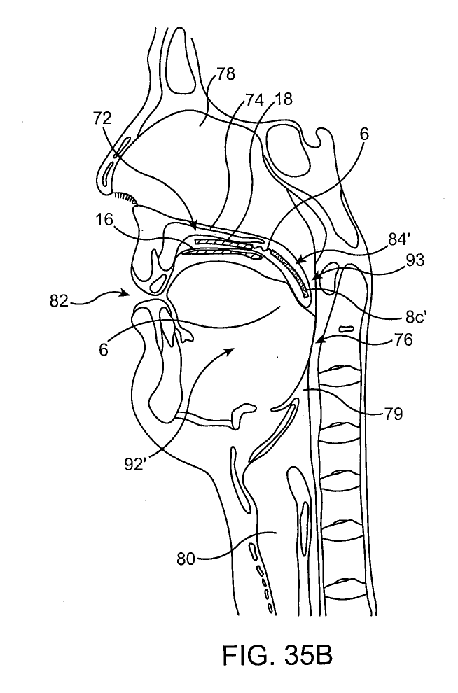

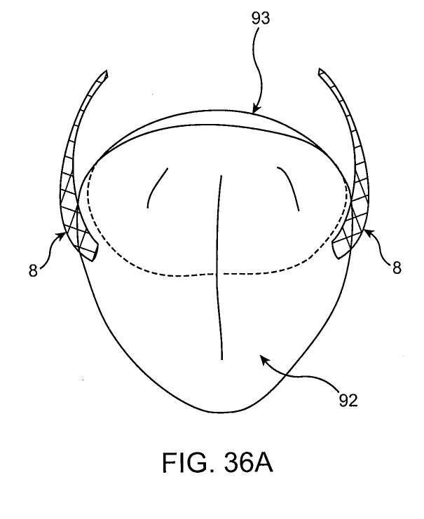

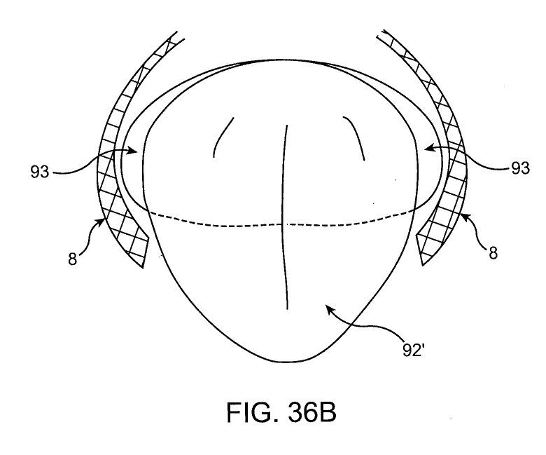

FIGS. 35 and 36 depict an embodiment of keeping the airways open with lateral wall implants. FIG. 35A shows a side view of a patient's face with a actuator element 8 located in the lateral wall of the airway. FIG. 35A depicts the tongue 92 in its normal state. FIG. 35B depicts the tongue 92′ in a collapsed state. When the tongue is in this state or before it goes into the collapsed state the actuator element 8 is energized so as to stretch the lateral walls and open the airway, as shown in FIG. 36B. FIGS. 36A and 36B are a view of the airway as seen through the mouth of patient. FIG. 36 A depicts the actuator elements 8 in a non-energized state and the tongue in a non-collapsed state. When the tongue collapses or it has a tendency to collapse, such as during sleep, the actuator element 8 is energized and airway walls are pushed away from the tongue and creates an open air passageway 93. This embodiment is particularly useful in obese patients.

### Airway Diseases

During sleep, the muscles in the roof of the mouth (soft palate), tongue and throat relax. If the tissues in the throat relax enough, they vibrate and may partially obstruct the airway. The more narrowed the airway, the more forceful the airflow becomes. Tissue vibration increases, and snoring grows louder. Having a low, thick soft palate or enlarged tonsils or tissues in the back of the throat (adenoids) can narrow the airway. Likewise, if the triangular piece of tissue hanging from the soft palate (uvula) is elongated, airflow can be obstructed and vibration increased. Being overweight contributes to narrowing of throat tissues. Chronic nasal congestion or a crooked partition between the nostrils (deviated nasal septum) may be to blame.

Snoring may also be associated with sleep apnea. In this serious condition, excessive sagging of throat tissues causes your airway to collapse, preventing breathing. Sleep apnea generally breaks up loud snoring with 10 seconds or more of silence. Eventually, the lack of oxygen and an increase in carbon dioxide signal causes the person to wake up, forcing the airway open with a loud snort.

Obstructive sleep apnea occurs when the muscles in the back of the throat relax. These muscles support the soft palate, uvula, tonsils and tongue. When the muscles relax, the airway is narrowed or closed during breathing in, and breathing is momentarily cut off. This lowers the level of oxygen in the blood. The brain senses this decrease and briefly rouses the person from sleep so that the airway can be reopened. Typically, this awakening is so brief that it cannot be remembered. Central sleep apnea, which is far less common, occurs when the brain fails to transmit signals to the breathing muscles.

Thus, it can be seen that airway disorders, such as sleep apnea and snoring, are caused by improper opening of the airway passageways. The devices and methods described herein are suitable for the treatment of disorders caused by the improper opening of the air passageways. The devices can be implanted in any suitable location such as to open up the airways. The opening of the passageways need not be a complete opening and in some conditions a partial opening is sufficient to treat the disorder.

In addition to air passageway disorders, the implants disclosed herein are suitable for use in other disorders. The disorders treated with the devices include those that are caused by improper opening and/or closing of passageways in the body, such as various locations of the gastro-intestinal tract or blood vessels. The implantation of the devices are suitable for supporting walls of passageways The devices can be implanted in the walls of the gastro-intestinal tract, such as the esophagus to treat acid reflux. The gastro-intestinal tract or blood vessel devices can be used in combination with the sensors described above. Also, the implants and/or sphincters can be used for disorders of fecal and urinary sphincters. Further, the implants of said invention can be tailored for specific patient needs.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

What is claimed is:

1. A device for powering an airway implant, comprising:
a mouthpiece;
a power transfer circuit attached to the mouthpiece, comprising:
a battery;
a receive circuit configured to receive a first inductive power transfer from an external device and to charge the battery with a current induced in the receive circuit by the external device, and
a transmit circuit configured to receive power from the battery and to provide a second inductive power transfer to the airway implant.

2. The device of claim 1 wherein the receive circuit comprises a pickup coil and the transmit circuit comprises a power transmission coil.

3. The device of claim 1, further comprising a timer coupled to the transmit and receive circuits and configured to activate the transmit circuit a predetermined time after the first inductive power transfer is complete.

4. The device of claim 1 further comprising a charge controller coupled to the receive circuit and the battery, wherein the charge controller is configured to deliver a charging current to the battery.

5. The device of claim 4 wherein the charge controller has a first operating mode in which the charging current is delivered to the battery at a substantially constant level, and a second operating mode in which the charging current is delivered to the battery so as to maintain the battery at a substantially constant voltage.

6. The device of claim 1 wherein the transmit circuit comprises a first resonator circuit coupled to a driver, and wherein the driver is configured to control an oscillation frequency of the first resonator circuit

7. The device of claim 1 wherein the mouthpiece comprises a dental retainer material.

8. The device of claim 1 wherein the battery comprises a lithium polymer battery.

9. A charging device for use with an airway implant system comprising a mouthpiece and an airway implant, the device comprising:
a housing adapted to receive the mouthpiece;
a power transfer circuit configured to source an inductive power transfer when the mouthpiece is received at the housing, the power transfer circuit comprising a power transmission coil;
a proximity detection circuit configured to detect a proximity of the mouthpiece to the power transmission coil and to generate an output signal based on said proximity, wherein the charging device initiates the inductive power transfer when a value of the output signal exceeds a predetermined threshold.

10. The charging device of claim 9 further comprising a microprocessor coupled to the power transfer and proximity detection circuits.

11. The charging device of claim 10 further comprising a timer, and wherein the microprocessor is configured to deactivate the power transfer circuit based on a value of the timer.

12. The charging device of claim 11 wherein the timer is configured to measure an interval comprising a charging interval and a cooling interval, and wherein the microprocessor is configured to deactivate the power transfer circuit upon expiration of the charging interval.

13. The charging device of claim 12 wherein the microprocessor is configured to activate and deactivate the power transfer circuit from time to time during the cooling interval.

14. The charging device of claim 12 further comprising a status indicator configured to indicate an operating state of the charging device.

15. The charging device of claim 14 wherein the status indicator signals a first operating state during the charging interval and a second operating state upon expiration of the cooling interval.

16. The charging device of claim 10 wherein the microprocessor is configured to issue commands to the mouthpiece via the power transmission coil.

17. The charging device of claim 10 wherein the microprocessor is configured to receive operating information from the mouthpiece.

18. The charging device of claim 17 further comprising a communications interface coupled to the microprocessor and configured to exchange data with an external device.

19. A device for powering an airway implant, comprising:
a non-implanted portion adapted and configured to be worn in a patient's mouth;
a power transfer circuit attached to the non-implanted portion, comprising:
a receive circuit configured to receive a first inductive power transfer from a charging device and to charge a battery with a current induced in the receive circuit by the charging device, and
a transmit circuit coupled to the battery and configured to provide a second inductive power transfer from the non-implanted portion to the airway implant; and
a first microprocessor coupled to the power transfer circuit and configured to control operation of the transmit and receive circuits.

20. The device of claim 19 wherein the receive circuit comprises a pickup coil and wherein the first microprocessor is configured to monitor a voltage level of the pickup coil.

21. The device of claim 20 wherein the first microprocessor is configured to communicate a voltage level of the battery using the transmit circuit in response to a request from the charging device.

22. The device of claim 19 wherein the first microprocessor is configured to activate or deactivate the power transfer circuit in response to one or more commands received from the charging device.

23. The device of claim 19 wherein the first microprocessor is configured to deactivate the transmit circuit a predetermined time after the first inductive power transfer is complete.

24. The device of claim 19 wherein the first microprocessor is configured to detect an ambient temperature of the device and to deactivate the power transfer circuit if the ambient temperature exceeds a predetermined threshold.

25. The device of claim 19 wherein the first microprocessor is configured to vary a power level of the second inductive power transfer.

26. The device of claim 25 wherein the first microprocessor varies the power level of the second inductive power transfer based on a proximity of the airway implant device.

27. The device of claim 25 wherein the first microprocessor varies the power level of the second inductive power transfer in response to one or more commands from the charging device.

28. The device of claim 25 wherein the first microprocessor varies the power level of the of the second inductive power transfer independent of a voltage of the battery.

29. The device of claim 25 wherein the first microprocessor is configured to communicate the power level of the second inductive power transfer to the charging device.

30. The device of claim 19 wherein the first microprocessor is configured to initialize a timer based on a voltage level of the pickup coil.

31. The device of claim 30 wherein the first microprocessor is configured to activate the transmit circuit upon expiration of the timer.

32. The device of claim 19 wherein the charging device comprises a second microprocessor.

33. The device of claim 32 wherein the charging device comprises a resonator circuit configured to source power for the first inductive power transfer, and wherein the second microprocessor is configured to control operation of the resonator circuit.

34. The device of claim 33 wherein the second microprocessor is configured to vary a power level of the resonator circuit.

35. The device of claim 33 wherein the second microprocessor varies the power level of the resonator circuit based upon a proximity of the non-implanted portion to the charging device.

36. The device of claim 33 wherein the charger further comprises a storage device configured to store event data including a time of the first inductive power transfer.

37. A method of powering an airway implant device having a non-implanted portion and an implant portion, the method comprising:
receiving a first inductive power transfer at the non-implanted portion;
charging a power source of the non-implanted portion with a current derived from the first inductive power transfer; and
performing a second inductive transfer from the non-implanted portion to the implant portion, wherein the power source supplies energy for the second inductive power transfer.

38. An inductively powering system comprising of an implanted receiver in the body cavity and/or tissue that is coupled to a prosthesis; and a wearable transmitter device that includes a power source.

39. An inductive power transfer system, comprising:
a prosthesis;
a receiver implanted in a body cavity and coupled to the prosthesis; and
a wearable transmitter, comprising:
a power source configured to supply an inductive power transfer to the receiver for operating the prosthesis, and
a timer coupled to the power source and configured to define an interval during which the first inductive power transfer is disabled.

40. A method of treating a patient suffering from sleep disordered breathing by placing an implant in the upper airway and inductively powering the implant by means of an implanted receiver and a wearable transmitter that includes a power source.
